SEQUENCE LISTING (99 sequences)

SEQ ID NO: 1 - DNA, 80 bp, Artificial Sequence
  Synthesized nucleic acid molecule referred to as OLI001
  misc_feature (34)..(58): n is a, c, g, or t
acactctttccctacacgacgctcttccgatctnnnnnnnnnnnnnnnnnnnnnnnnnagatcggaagagcacacgtctg

SEQ ID NO: 2 - DNA, 100 bp, Artificial Sequence
  Synthesized nucleic acid molecule referred to as OLI002
  misc_feature (4)..(78): n is a, c, g, or t
atcnnnnnnnnnnnnnnnnnnnnnnnnnnnnnnnnnnnnnnnnnnnnnnnnnnnnnnnnnnnnnnnnnnnnnnnnnnncagacgtgtgctcttccgatct

SEQ ID NO: 3 - DNA, 22 bp, Artificial Sequence
  Synthesized nucleic acid molecule referred to as OLI004
agatcggaagagcacacgtctg

SEQ ID NO: 4 - DNA, 100 bp, Artificial Sequence
  Synthesized nucleic acid molecule referred to as OLI005
  misc_feature (4)..(83): n is a, c, g, or t
gggnnnnnnnnnnnnnnnnnnnnnnnnnnnnnnnnnnnnnnnnnnnnnnnnnnnnnnnnnnnnnnnnnnnnnnnnnnnnnnnngcaatctatctacaagg

SEQ ID NO: 5 - DNA, 17 bp, Artificial Sequence
  Synthesized nucleic acid molecule referred to as OLI005
ccttgtagatagattgc

SEQ ID NO: 6 - DNA, 100 bp, Artificial Sequence
  Synthesized nucleic acid molecule referred to as OLI006
  misc_feature (4)..(21): n is a, c, g, or t
  misc_feature (40)..(83): n is a, c, g, or t
ctgnnnnnnnnnnnnnnnnnngcgatcgcttgcggccgcnnnnnnnnnnnnnnnnnnnnnnnnnnnnnnnnnnnnnnnnnnnnccttgtagatagattgc

SEQ ID NO: 7 - DNA, 17 bp, Artificial Sequence
  Synthesized nucleic acid molecule referred to as OLI007
gcaatctatctacaagg

SEQ ID NO: 8 - DNA, 99 bp, Artificial Sequence
  Synthesized nucleic acid molecule referred to as OLI008
  misc_feature (4)..(82): n is a, c, g, or t
atcnnnnnnnnnnnnnnnnnnnnnnnnnnnnnnnnnnnnnnnnnnnnnnnnnnnnnnnnnnnnnnnnnnnnnnnnnnnnnnncgtaagttctatgcttg

SEQ ID NO: 9 - DNA, 17 bp, Artificial Sequence
  Synthesized nucleic acid molecule referred to as OLI009
caagcatagaacttacg

SEQ ID NO: 10 - DNA, 100 bp, Artificial Sequence
  Synthesized nucleic acid molecule referred to as OLI010
  misc_feature (4)..(83): n is a, c, g, or t
ctgnnnnnnnnnnnnnnnnnnnnnnnnnnnnnnnnnnnnnnnnnnnnnnnnnnnnnnnnnnnnnnnnnnnnnnnnnnnnnnnncaagcatagaacttacg

SEQ ID NO: 11 - DNA, 34 bp, Artificial Sequence
  Synthesized nucleic acid molecule referred to as OLI011
cgtaagttctatgcttgcgtaagttctatgcttg

SEQ ID NO: 12 - DNA, 100 bp, Artificial Sequence
  Synthesized nucleic acid molecule referred to as OLI012
  misc_feature (4)..(82): n is a, c, g, or t
atcnnnnnnnnnnnnnnnnnnnnnnnnnnnnnnnnnnnnnnnnnnnnnnnnnnnnnnnnnnnnnnnnnnnnnnnnnnnnnnngtacgaacgcttcagacc

SEQ ID NO: 13 - DNA, 18 bp, Artificial Sequence
  Synthesized nucleic acid molecule referred to as OLI013
ggtctgaagcgttcgtac

SEQ ID NO: 14 - DNA, 24 bp, Artificial Sequence
  Synthesized nucleic acid molecule referred to as OLI014
acactctttccctacacgacgctc

SEQ ID NO: 15 - DNA, 49 bp, Artificial Sequence
  Synthesized nucleic acid molecule referred to as OLI015
gtacgaacgcttcagaccagcgctgcaggcgtaatcatggtcatagctg

SEQ ID NO: 16 - DNA, 50 bp, Artificial Sequence
  Synthesized nucleic acid molecule referred to as OLI016
gagcgtcgtgtagggaaagagtgtagatctactggccgtcgttttacaac

SEQ ID NO: 17 - DNA, 68 bp, Artificial Sequence
  Synthesized nucleic acid molecule referred to as OLI017
gtcgacagcgctctgcaggcgtaatcatggtcatagctgtttcctgtgtgaaattgttatccgctcac

SEQ ID NO: 18 - DNA, 49 bp, Artificial Sequence
  Synthesized nucleic acid molecule referred to as OLI020
attacccgccctttcgtctcgcggaaatatccattacccacagtaagac

SEQ ID NO: 19 - DNA, 68 bp, Artificial Sequence
  Synthesized nucleic acid molecule referred to as OLI021
gcctgcagagcgctgtcgacgtatcacgaggccagcttttcaagaggcatcgcacagttttagcgagg

SEQ ID NO: 20 - DNA, 52 bp, Artificial Sequence
  Synthesized nucleic acid molecule referred to as OLI022
cgcgagacgaaagggcgggtaatatcatacgtaatgctcaaccttaagctgc

SEQ ID NO: 21 - DNA, 35 bp, Artificial Sequence
  Synthesized nucleic acid molecule referred to as OLI023
ccatatgccaaattgagggacccaaatgttatttc

SEQ ID NO: 22 - DNA, 35 bp, Artificial Sequence
  Synthesized nucleic acid molecule referred to as OLI024
gaaataacatttgggtccctcaatttggcatatgg

SEQ ID NO: 23 - DNA, 58 bp, Artificial Sequence
  Synthesized nucleic acid molecule referred to as OLI025
gtaaaacgacggccacctaggtatatataaccatcaagtaatacatatttaacttagc

SEQ ID NO: 24 - DNA, 51 bp, Artificial Sequence
  Synthesized nucleic acid molecule referred to as OLI026
cctaggtggccgtcgttttacaacgtcgtgactgggaaaaccctggcgtta

SEQ ID NO: 25 - DNA, 55 bp, Artificial Sequence
  Synthesized nucleic acid molecule referred to as OLI027
gttgtaaaacgacggccacctaggagcgctgcactcttcttctgaagagttaagc

SEQ ID NO: 26 - DNA, 35 bp, Artificial Sequence
  Synthesized nucleic acid molecule referred to as OLI028
agatctgcctagaagcagaattcggtaagcgttgg

SEQ ID NO: 27 - DNA, 57 bp, Artificial Sequence
  Synthesized nucleic acid molecule referred to as OLI029
cgcctgcagagcgctgtcgacggatccagtgcgaatttttctgaatcgaatggtagg

SEQ ID NO: 28 - DNA, 33 bp, Artificial Sequence
  Synthesized nucleic acid molecule referred to as OLI030
gaattctgcttctaggcagatctgacgatcacc

SEQ ID NO: 29 - DNA, 39 bp, Artificial Sequence
  Synthesized nucleic acid molecule referred to as OLI033
ctgcagagcgctgtcgacgtatcacgaggccagcttttc

SEQ ID NO: 30 - DNA, 28 bp, Artificial Sequence
  Synthesized nucleic acid molecule referred to as OLI034
cgcgagacgaaagggcgggtaataactg

SEQ ID NO: 31 - DNA, 23 bp, Artificial Sequence
  Synthesized nucleic acid molecule referred to as OLI035
cgcgagacgaaagggcgggtaat

SEQ ID NO: 32 - DNA, 24 bp, Artificial Sequence
  Synthesized nucleic acid molecule referred to as OLI036
ccagtgagcccgcttgcagagttc

SEQ ID NO: 33 - DNA, 24 bp, Artificial Sequence
  Synthesized nucleic acid molecule referred to as OLI037
cattttggtagccaagatagaatg

SEQ ID NO: 34 - DNA, 24 bp, Artificial Sequence
  Synthesized nucleic acid molecule referred to as OLI038
catatttgagtacgatcgttcctg

SEQ ID NO: 35 - DNA, 24 bp, Artificial Sequence
  Synthesized nucleic acid molecule referred to as OLI039
caacccacgtggtttagcaacacc

SEQ ID NO: 36 - DNA, 44 bp, Artificial Sequence
  Synthesized nucleic acid molecule referred to as OLI040
gctggcctcgtgatacgtcgacacactctttccctacacgacgc

SEQ ID NO: 37 - DNA, 49 bp, Artificial Sequence
  Synthesized nucleic acid molecule referred to as OLI041
gcttaactcttcagaagaagagtgcagcgctggtctgaagcgttcgtac

SEQ ID NO: 38 - DNA, 31 bp, Artificial Sequence
  Synthesized nucleic acid molecule referred to as OLI042
agcgctgcactcttcttctgaagagttaagc

SEQ ID NO: 39 - DNA, 48 bp, Artificial Sequence
  Synthesized nucleic acid molecule referred to as OLI043
gaaacagctatgaccatgattacgcctgcagttcattcggataattgg

SEQ ID NO: 40 - DNA, 49 bp, Artificial Sequence
  Synthesized nucleic acid molecule referred to as OLI044
cgattcagaaaaattcgcactggatccacactctttccctacacgacgc

SEQ ID NO: 41 - DNA, 48 bp, Artificial Sequence
  Synthesized nucleic acid molecule referred to as OLI045
gaaacagctatgaccatgattacgcctgcaggtctgaagcgttcgtac

SEQ ID NO: 42 - DNA, 50 bp, Artificial Sequence
  Synthesized nucleic acid molecule referred to as OLI059
tctttccctacacgacgctcgcgatcgcggattagaagccgccgagcggg

SEQ ID NO: 43 - DNA, 50 bp, Artificial Sequence
  Synthesized nucleic acid molecule referred to as OLI060
ctttgtacaagaaagctgggtcattaggcctgaagcctgcttttttgtac

SEQ ID NO: 44 - DNA, 50 bp, Artificial Sequence
  Synthesized nucleic acid molecule referred to as OLI061
gtacaaaaaagcaggcttcaggcctaatgacccagctttcttgtacaaag

SEQ ID NO: 45 - DNA, 25 bp, Artificial Sequence
  Synthesized nucleic acid molecule referred to as OLI062
gcaaattaaagccttcgagcgtccc

SEQ ID NO: 46 - DNA, 69 bp, Artificial Sequence
  Synthesized nucleic acid molecule referred to as OLI063
cgctcgaaggctttaatttgcaaagaatacgttattctttcatcaaccagcttttcaattcaattcatc

SEQ ID NO: 47 - DNA, 70 bp, Artificial Sequence
  Synthesized nucleic acid molecule referred to as OLI064
gtctgaagcgttcgtacgcggccgcaaagaataacgtattctttcatcaagctctaatttgtgagtttag

SEQ ID NO: 48 - DNA, 48 bp, Artificial Sequence
  Synthesized nucleic acid molecule referred to as OLI065
gtaaaacgacggccagtgaattcacacaccatagcttcaaaatgtttc

SEQ ID NO: 49 - DNA, 33 bp, Artificial Sequence
  Synthesized nucleic acid molecule referred to as OLI066
tttgtaattaaaacttagattagattgctatgc

SEQ ID NO: 50 - DNA, 51 bp, Artificial Sequence
  Synthesized nucleic acid molecule referred to as OLI067
ctaagttttaattacaaacttttcgtttggatctttggataaggcagattg

SEQ ID NO: 51 - DNA, 70 bp, Artificial Sequence
  Synthesized nucleic acid molecule referred to as OLI068
cgttctcttttcaataaatttagtgataaaatattcacatacctaatgctctaaaggtgaagaattattc

SEQ ID NO: 52 - DNA, 70 bp, Artificial Sequence
  Synthesized nucleic acid molecule referred to as OLI069
aatttattgaaaagagaacgttactaacagtggaatttttttttctagatctaaaggtgaagaattattc

SEQ ID NO: 53 - DNA, 50 bp, Artificial Sequence
  Synthesized nucleic acid molecule referred to as OLI070
ctattaaaaaaaataaataggcttttcgtttggatctttggataaggcag

SEQ ID NO: 54 - DNA, 30 bp, Artificial Sequence
  Synthesized nucleic acid molecule referred to as OLI071
cctatttattttttttaatagttatgttag

SEQ ID NO: 55 - DNA, 53 bp, Artificial Sequence
  Synthesized nucleic acid molecule referred to as OLI072
gaccatgattacgccaagctgcagaacctttagaggtaataagagataaatac

SEQ ID NO: 56 - DNA, 52 bp, Artificial Sequence
  Synthesized nucleic acid molecule referred to as OLI073
gtaaaacgacggccagtgaattcgtattctgataaatctaaagagaaattac

SEQ ID NO: 57 - DNA, 57 bp, Artificial Sequence
  Synthesized nucleic acid molecule referred to as OLI074
aacttttctttttttttttggtggcattggcacaaatgtggtttcctttcttattgc

SEQ ID NO: 58 - DNA, 53 bp, Artificial Sequence
  Synthesized nucleic acid molecule referred to as OLI075
atgccaccaaaaaaaaaaagaaaagtttctaaaggtgaagaattattcactgg

SEQ ID NO: 59 - DNA, 49 bp, Artificial Sequence
  Synthesized nucleic acid molecule referred to as OLI076
gaccatgattacgccaagcttgccggtagaggtgtggtcaataagagcg

SEQ ID NO: 60 - DNA, 70 bp, Artificial Sequence
  Synthesized nucleic acid molecule referred to as OLI082
atgagctgcaggcctgccatgctgaggggttgcaaagcagggcttttcatgaagcctgcttttttgtaca

SEQ ID NO: 61 - DNA, 70 bp, Artificial Sequence
  Synthesized nucleic acid molecule referred to as OLI083
gaagagccctccgaagcctcaaagctaatcaacctcaggttcccaatagctgacccagctttcttgtaca

SEQ ID NO: 62 - DNA, 70 bp, Artificial Sequence
  Synthesized nucleic acid molecule referred to as OLI084
gggggcggcggcgcaggaggtgcaagtgcggggccggctcccgagtacatgaagcctgcttttttgtaca

SEQ ID NO: 63 - DNA, 70 bp, Artificial Sequence
  Synthesized nucleic acid molecule referred to as OLI085
ccaaggcggtccaggttcaccaagacactctgcgcaccatgtactttgcttgacccagctttcttgtaca

SEQ ID NO: 64 - DNA, 70 bp, Artificial Sequence
  Synthesized nucleic acid molecule referred to as OLI086
ggcagcccgcagcccgtggtagtgccggagccttgctcctcttcactcatgaagcctgcttttttgtaca

SEQ ID NO: 65 - DNA, 70 bp, Artificial Sequence
  Synthesized nucleic acid molecule referred to as OLI087
aggctgcccgccgtgccctgcagtacctcaagatcatggcaggcagcaagtgacccagctttcttgtaca

SEQ ID NO: 66 - DNA, 20 bp, Artificial Sequence
  Synthesized nucleic acid molecule referred to as OLI1126
agagttgccccagaagaaca

SEQ ID NO: 67 - DNA, 20 bp, Artificial Sequence
  Synthesized nucleic acid molecule referred to as OLI1127
ggcttggatggaaacgtaga

SEQ ID NO: 68 - DNA, 26 bp, Artificial Sequence
  Synthesized nucleic acid molecule referred to as pPCRsplitGFPFw
tccaaagatccaaacgaaaagagaga

SEQ ID NO: 69 - DNA, 27 bp, Artificial Sequence
  Synthesized nucleic acid molecule referred to as pPCRsplitGFPRv
gcgccttatttgtacaattcatccata

SEQ ID NO: 70 - DNA, 60 bp, Artificial Sequence
  Synthesized nucleic acid molecule referred to as siGFP01
tctaaaggtgaagaattattcactggtgttgtcccaattttggttgaattagatggtgat

SEQ ID NO: 71 - DNA, 60 bp, Artificial Sequence
  Synthesized nucleic acid molecule referred to as siGFP02
agatggtgatgttaatggtcacaaattttctgtctccggtgaaggtgaaggtgatgctac

SEQ ID NO: 72 - DNA, 60 bp, Artificial Sequence
  Synthesized nucleic acid molecule referred to as siGFP03
gtgatgctacttacggtaaattgaccttaaaatttatttgtactactggtaaattgccag

SEQ ID NO: 73 - DNA, 60 bp, Artificial Sequence
  Synthesized nucleic acid molecule referred to as siGFP04
aaattgccagttccatggccaaccttagtcactactttaacttatggtgttcaatgtttt

SEQ ID NO: 74 - DNA, 60 bp, Artificial Sequence
  Synthesized nucleic acid molecule referred to as siGFP05
tcaatgtttttctagatacccagatcatatgaaacaacatgactttttcaagtctgccat

SEQ ID NO: 75 - DNA, 60 bp, Artificial Sequence
  Synthesized nucleic acid molecule referred to as siGFP06
agtctgccatgccagaaggttatgttcaagaaagaactatttttttcaaagatgacggta

SEQ ID NO: 76 - DNA, 60 bp, Artificial Sequence
  Synthesized nucleic acid molecule referred to as siGFP07
gatgacggtaactacaagaccagagctgaagtcaagtttgaaggtgataccttagttaat

SEQ ID NO: 77 - DNA, 60 bp, Artificial Sequence
  Synthesized nucleic acid molecule referred to as siGFP08
cttagttaatagaatcgaattaaaaggtattgattttaaagaagatggtaacattttagg

SEQ ID NO: 78 - DNA, 60 bp, Artificial Sequence
  Synthesized nucleic acid molecule referred to as siGFP09
acattttaggtcacaaattggaatacaactataactctcacaatgtttacatcatggctg

SEQ ID NO: 79 - DNA, 60 bp, Artificial Sequence
  Synthesized nucleic acid molecule referred to as siGFP10
atcatggctgacaaacaaaagaatggtatcaaagttaacttcaaaattagacacaacatt

SEQ ID NO: 80 - DNA, 60 bp, Artificial Sequence
  Synthesized nucleic acid molecule referred to as siGFP11
acacaacattgaagatggttctgttcaattagctgaccattatcaacaaaatactccaat

SEQ ID NO: 81 - DNA, 60 bp, Artificial Sequence
  Synthesized nucleic acid molecule referred to as siGFP12
atactccaattggtgatggtccagtcttgttaccagacaaccattacttatccactcaat

SEQ ID NO: 82 - DNA, 60 bp, Artificial Sequence
  Synthesized nucleic acid molecule referred to as siGFP13
ttaccagacaaccattacttatccactcaatctgccttatccaaagatccaaacgaaaag

SEQ ID NO: 83 - DNA, 619 bp, Artificial Sequence
  Synthesized nucleic acid molecule referred to as BC1
agatctacactctttccctacacgacgctcttccgatctgttcgtgccttagttgtttatggtgagatcggaagagcacacgtctgtaagcaagctgcgacaggaaagatccctcttaccaaaagacaactcggcgaacttcggctggggtccctagttcttcccttggccggtttttacggggatagcctcggctcactgcttatcattcgtctatttcctaattgcgcaatctatctacaagggtttccgctaattgagactataggatgtttcgcgccatatggtcgcggccgcaagcgatcgccaacaaaatctaattacccagatccttgttcccgttgaatctcggttacgcccggacgcgtccgcatcaggtcgcttatccggattgcagttaatgagctaaacgtaagttctatgcttgactcttaccatagtgcacagggcgctaccgaaaaaaatcgaacggcgggcgtaatcagaacgctacggcggggtatattccagatcctattggaggaatgtgctgtcggtgtgatgtagtggagccgaatatgtaggttatctgtttgtctgtctggttgtgggcgtacgaacgcttcagaccagcgctgca

SEQ ID NO: 84 - DNA, 620 bp, Artificial Sequence
  Synthesized nucleic acid molecule referred to as BC2
agatctacactctttccctacacgacgctcttccgatctctttttcgtatgttggtgcctatttagatcggaagagcacacgtctgtcggcccggacgacggctgcaccaaaaagaacccaggaccccacatccgaagctttccagagcaggcatcaagccgatgcgtgtgggtcttgctcttgtgttatttcgtgccggggtctgttttgtggtcgggtgatgagctcagcaatctatctacaaggaccatacggaaaccaagacttttgatcagaccggggccgacgttgcggccgcaagcgatcgcttatccaccataatcagacagatccaggtgccgtgtcacgttacaggtttcgggtaggaactggcctgttgtccgtgtaggcctgttacattcacagcacatccgtaagttctatgcttgacattagcgccctaattaagacacaactacattccaatcagcattgcagcggaacacgaaagggcctagccaattaggagcagtcgtatcatttggtctggggtggtaacttgggttgagcttattttgtgttgagactgtagcgggtggtagcatgggatgatgtacgaacgcttcagaccagcgctgca

SEQ ID NO: 85 - DNA, 622 bp, Artificial Sequence
  Synthesized nucleic acid molecule referred to as BC3
agatctacactctttccctacacgacgctcttccgatcttcttagtggtctttccactctttggagatcggaagagcacacgtctgcctgccacaaactgacgtaacataaagccatatacgctccagatgcatagaggcgcgctcccagaaaaatagcaggatggggactgtgggtttgggaagtggatatcctcgctaggtcgtctcggtcccgtctcagatttagcgtatgtaccatgatgtttgcaatctatctacaaggataataaacgttatcccctaatacgttgacggagatggaccccagcggccgcaagcgatcgccaatcaacaggtagaaaccatcggcgtttatcttgggttgcagtgtgttttcgctcgcttgttggcttgtcagggtgtgcggactccaacagtctccaatacgtaagttctatgcttgtatccgtctggcccgagagccgaaaatgtcgaccactatccacacagccctcacacaagttcagaacagatctcatccttgagattccagactaaggagtgtaatcgcggtatgctatttggcaaatctgtcccataattggcctggaaccgtacgaacgcttcagaccagcgctgca

SEQ ID NO: 86 - DNA, 28 bp, Artificial Sequence
  Synthesized nucleic acid molecule referred to as TM3
atctcgagtcccataactaacctaccat

SEQ ID NO: 87 - DNA, 28 bp, Artificial Sequence
  Synthesized nucleic acid molecule referred to as TM4
acggtaccagtaaatggcagtttctagg

SEQ ID NO: 88 - DNA, 30 bp, Artificial Sequence
  Synthesized nucleic acid molecule referred to as TM-Hind
ccccaagcttacctaccgaccaactttcat

SEQ ID NO: 89 - DNA, 31 bp, Artificial Sequence
  Synthesized nucleic acid molecule referred to as TM-Sph
cacatgcatgctttagcataggaagccaaga

SEQ ID NO: 90 - DNA, 28 bp, Artificial Sequence
  Synthesized nucleic acid molecule referred to as TM7
cacatgcatgcagcttttcaattcatct

SEQ ID NO: 91 - DNA, 30 bp, Artificial Sequence
  Synthesized nucleic acid molecule referred to as TM8
acgcgtcgacatcattacgaccgagattcc

SEQ ID NO: 92 - DNA, 24 bp, Artificial Sequence
  Synthesized nucleic acid molecule referred to as Primer MN2503
gaggctataccaccacctcctttc

SEQ ID NO: 93 - DNA, 43 bp, Artificial Sequence
  Synthesized nucleic acid molecule referred to as Primer MN2509
ggaattatattttgagtcgacccatctaatgtactggtatcac

SEQ ID NO: 94 - DNA, 45 bp, Artificial Sequence
  Synthesized nucleic acid molecule referred to as Primer MN2510
ctactcctcttccgttttcagaattcagatggaccagccaaggac

SEQ ID NO: 95 - DNA, 50 bp, Artificial Sequence
  Synthesized nucleic acid molecule referred to as Primer MN2511
gagtagggaatattactggctgcagataagtcttgaatgaacgtatacgc

SEQ ID NO: 96 - DNA, 49 bp, Artificial Sequence
  Synthesized nucleic acid molecule referred to as Primer MN2512
ggcaaaaaaagaaaagctccggatccagattgtacgtaaggtgacaagc

SEQ ID NO: 97 - DNA, 46 bp, Artificial Sequence
  Synthesized nucleic acid molecule referred to as Primer MN2515
ttggctggtccatctgaattcgcgatgatgttccgatcggaagctc

SEQ ID NO: 98 - DNA, 42 bp, Artificial Sequence
  Synthesized nucleic acid molecule referred to as Primer MN2517
gaggtggtggtatagcctcgatgcacagaaactaaggtacaa

SEQ ID NO: 99 - DNA, 44 bp, Artificial Sequence
  Synthesized nucleic acid molecule referred to as Primer MN2518
tcattcaagacttatctgcaggctctgctaactcagtcatattg

(19) United States
(12) Patent Application Publication
(10) Pub. No.: US 2024/0294944 A1
(43) Pub. Date: Sep. 5, 2024

(54) Artificial Chromosome Vector and Use Thereof

(71) Applicant: The University of Tokyo, Tokyo (JP)

(72) Inventors: Takehiko Kobayashi, Tokyo (JP); Tetsushi Iida, Tokyo (JP)

(21) Appl. No.: 17/909,341

(22) PCT Filed: Mar. 9, 2021
(86) PCT No.: PCT/JP2021/009232

§ 371 Date: Nov. 25, 2022

(30) Foreign Application Priority Data
Mar. 9, 2020 (JP) 2020-039614

(51) Int. Cl. C12N 15/90; C12N 15/81; C12N 15/85; C12N 15/88; C12Q 1/6809

(52) U.S. Cl. CPC C12N 15/90; C12N 15/81; C12N 15/85; C12N 15/88; C12Q 1/6809; C12Q 2600/158

(57) ABSTRACT

Provided is an artificial chromosome vector derived from a chromosome of a eukaryote, the vector comprising repetitive units of multiple ribosomal RNA genes (rDNA) and intergenic regions between the repetitive units, wherein an intergenic region 1 (IGS1) of the intergenic regions comprises a barcode sequence for incorporating and distinguishing a target gene.

### REFERENCE TO A SEQUENCE LISTING SUBMITTED VIA EFS-WEB

The content of the ASCII text file of the sequence listing named “20230126_034574_036US1_subseq_ST25” which is 27,408 bytes in size was created on Jan. 26, 2023 and electronically submitted via EFS-Web is incorporated herein by reference in its entirety.

### TECHNICAL FIELD

The present invention generally relates to an artificial chromosome vector and to a use therefor.

### BACKGROUND ART

In the composition of human genome, roughly half of the total of both large and small genome is occupied by repetitive sequences. These repetitive sequences comprise ribosomal RNA genes (rDNA). Ribosomes are the most abundant protein-RNA complexes in cells, and genes thereof, i.e., ribosomal RNA genes (rDNA), are also found in multiple copies (rDNA repeats).

In the case of budding yeasts, for example, about 150 copies are tandemly repeated on the 12th chromosome, constituting about 10% of the total genome. Ribosomes are essential translation mechanisms in all living organisms, so mechanisms for maintaining multiple copies of rDNA are also essential. In fact, even if the rDNA of a budding yeast is artificially reduced to two copies, it is then restored to 150 copies. That is, two copies (18 kb) are elongated to 150 copies (1.4 Mb). This recovery of copy numbers by amplification is also observed in other organisms.

As a cassette for multiple gene insertion on rDNA, WO 2016/027943 A1 discloses a cassette comprising an rDNA NTS (nontranscript sequence) N-terminal fragment gene, an insertion gene, an auxotrophic selection marker gene comprising a promoter region, and a Saccharomyces cerevisiae rDNA NTS C-terminal fragment gene, in that sequence.

### CITATION LIST

### Patent Document

Patent Document 1: WO 2016/027943 A1

### SUMMARY

### Technical Problem

In the cassette for multiple gene insertion described in WO 2016/027943 A1, the target gene is inserted into the intergenic region 2 (IGS2) of the NTS (FIG. 1a, etc.). However, a binding sequence for cohesin, a factor involved in rDNA stability, is present at the IGS2 insertion site, and, when a target gene is inserted into IGS2, this binding sequence may be damaged by the insertion.

It is an object of the present invention to provide an artificial chromosome vector capable of incorporating multiple genes, as well as novel applications using the artificial chromosome.

### Solution to Problem

Taking advantage of the specificity of the rDNA chromosomal function, the inventors perfected an artificial chromosome vector capable of incorporating multiple genes. The inventors have also discovered that novel applications such as an artificial gene amplification system are possible by using such an artificial chromosome vector, thereby perfecting the present invention.

That is, the invention of the present application encompasses the following inventions.
[1] An artificial chromosome vector derived from a eukaryotic chromosome, the vector comprising repetitive units of multiple ribosomal RNA genes (rDNA) and intergenic regions between the repetitive units, wherein an intergenic region 1 (IGS1) of the intergenic regions comprises a barcode sequence for incorporating and distinguishing a target gene.
[2] The artificial chromosome vector according to [1], wherein the intergenic region comprises an autonomously replicating sequence (ARS), a cohesin binding site, a 5S rDNA sequence, a non-coding promoter (E-pro) sequence and a replication fork barrier (RFB) sequence in that order from the 5′ side of the rDNA gene.
[3] The artificial chromosome vector according to [2], wherein the intergenic region further comprises a condensin binding site.
[4] The artificial chromosome vector according to any of [1] to [3], wherein each barcode sequence is inserted into a restriction enzyme site located between a 5S rDNA sequence and an E-pro sequence.
[5] The artificial chromosome vector according to any of [1] to [4], further comprising a telomere sequence and a centromere sequence.
[6] The artificial chromosome vector according to any of [1] to [3], wherein each barcode sequence comprises a promoter sequence and a terminator sequence for controlling expression of the target gene.
[7] The artificial chromosome vector according to [6], wherein the terminator sequence is disposed on the 3′ side of each target gene and the promoter sequence is disposed on the 5′ side.
[8] The artificial chromosome vector according to any of [1] to [7], wherein the number of repetitive units is from 2 to 150.
[9] The artificial chromosome vector according to any of [1] to [8], wherein the eukaryote is a human or yeast.
[10] The artificial chromosome vector according to [9], wherein the yeast is a budding yeast, and the repetitive units of rDNA are derived from chromosome 12 of the budding yeast.
[11] The artificial chromosome vector according to any of [6] to [10], wherein the promoter is a galactose inducible (GAL) promoter.
[12] The artificial chromosome vector according to any of [1] to [11], wherein the inserted target gene is homologous or heterologous to the host into which the artificial chromosome vector is introduced.
[13] The artificial chromosome vector according to any of [1] to [12], wherein the target genes to be inserted into the barcode sequences are identical or different from each other.
[14] A eukaryotic cell comprising the artificial chromosome vector according to any of [1] to [13].

[15] A method for manufacturing the artificial chromosome vector according to any of [1] to [13], the method comprising a step of inserting barcode sequences into IGS1.
[16] The method according to [15], wherein the step of inserting the barcode sequence comprises 1) a step of inserting a first barcode sequence and a selection marker sequence into a first IGS1 by homologous recombination between rDNA and a non-rDNA gene region; and 2) a step of inserting a second barcode sequence and a selection marker sequence different from the selection marker sequence of the 1) into a second IGS1 by homologous recombination between the first barcode sequence inserted in the 1) and a non-rDNA gene region.
[17] The method according to [16], comprising 3) a step of inserting a third barcode sequence and a selection marker sequence different from the selection marker sequence of the 2) into a third IGS1 by homologous recombination between the second barcode sequence inserted in the 2) and a non-rDNA gene region, wherein a fourth and subsequent barcode sequences may optionally be inserted into a fourth and subsequent IGS1s by repeating the same step as in the 3).
[18] The method according to [17], wherein the homologous recombination is achieved using homologous recombination vectors comprising each barcode sequence and selection marker sequence.
[19] The method according to [18], wherein the selection marker sequence is a uracil synthesis gene or lysine synthesis gene.
[20] A method for manufacturing multiple identical or different target genes or expression products of the target genes, the method comprising: 1) a step of inserting the target genes into the barcode sequences of the artificial chromosome vector according to any of [1] to [13], and 2) a step of maintaining and culturing in a host the resulting artificial chromosome vector obtained in the 1).
[21] The method according to [20], wherein the same target genes are amplified within the host.
[22] The method according to or [21], wherein the target genes are heterologous to the host.
[23] The method according to any of to [22], wherein the host is an rDNA unstable strain.
[24] The method according to [23], wherein the rDNA unstable strain lacks a CTF4 gene or RTT109 gene.
[25] The method according to any of to [24], wherein step 2) comprises a step of controlling an amount of expression of the target gene or an expression product by expressing or suppressing a Fob1 protein produced by the host.
[26] A method for analyzing behaviors of multiple different target genes derived from heterologous cells, or the expression products of the target genes, in host cells, the method comprising: 1) a step of maintaining and culturing an artificial chromosome vector according to any of [1] to [13], which is an artificial chromosome comprising a target gene inserted into a barcode sequence, in host cells, and 2) a step of analyzing a function of the target gene or an expression product thereof, or behaviors thereof in the host cells.
[27] The method according to [26], wherein an expression level of the expression product is measured in step 2).
[28] The method according to [27], wherein measuring of the expression level comprises measuring an amount of a transcription product or a translation product of the target gene.
[29] The method according to any of to [28], wherein step 2) is performed in the presence or absence of a substance that activates or inhibits the target gene or an expression product thereof.
[30] The method according to [29], wherein the substance is homologous or heterologous to the host.
[31] The method according to any of to [30], wherein step 2) comprises a comparison with a host into which the artificial chromosome vector has not been introduced.
[32] The method according to any of to [31], wherein the target gene is a drug resistance gene, the rDNA of the artificial chromosome vector is derived from a human, and the host is a yeast.
[33] An artificial cell comprising a liposome and the artificial chromosome vector according to any of [1] to [13].
[34] A method for manufacturing an artificial cell, the method comprising a step of introducing the artificial chromosome vector according to any of [1] to into a liposome.

### Advantageous Effects of Invention

With the present invention, an artificial chromosome vector that is useful for various purposes can be provided by using rDNA amplification and maintenance mechanisms. For example, while existing systems using plasmids are hampered by limitations on the number of genes that can be inserted and by loss of plasmid stability (maintenance rates) depending on the size and properties of the inserted genes, multiple genes can be introduced and maintained very stably by using an artificial chromosome vector having a stability maintenance mechanism.

Gene amplification is a method for producing large quantities of proteins, but has not been implemented due to the difficulty of control in in vivo systems using host cells. By using an artificial chromosome vector encoding the same gene, it is possible to construct a system (in vivo PCR) for continuously synthesizing the protein encoded by the gene in host cells, or in other words a mass production system for useful proteins and the like. Furthermore, this system is highly efficient because it uses a natural amplification system, and amplification can be controlled by turning on and off the Fob1 protein, which inhibits replication and induces DNA double-strand break. Unlike conventional mass production systems using multi-copy plasmids, moreover, it can stably maintain the amplified genes on the chromosome without the need for drug selection or the like.

Mass production of RNA is possible with the artificial chromosome vector of the invention.

By expressing the artificial chromosome vector in yeasts, which are model organisms relatively close to humans, it is possible to construct an “in-yeast” experimental system, which is a novel assay system intermediate between in vitro and in vivo. An “in-yeast” experimental system is an experimental method whereby a group of genes involved in a single reaction in a different organism are cloned to a yeast-derived artificial chromosome vector, and an exogenous reaction system is operated in a yeast to perform functional analysis such as identification of necessary factors. With the artificial chromosome vector of the invention, it is possible to construct a novel analysis model using such a heterologous reaction system.

For example, a yeast having a human protein production system could be prepared and used for drug discovery and analyzing drug targets, or to reconstruct a human repair system in the yeast, or for analyzing the mechanisms of cancer occurrence or cell aging. Research that is conventionally performed in mice and other individual animals could also potentially be performed in yeasts.

### BRIEF DESCRIPTION OF DRAWINGS

FIG. 1 is a schematic diagram of rDNA amplification and maintenance mechanisms.

FIG. 2 is a schematic diagram of an rDNA amplification model.

FIG. 3 is a schematic diagram of the structure of budding yeast rDNA.

FIG. 4 shows one example of a method for preparing an artificial chromosome vector.

FIG. 5 shows one example of a method for preparing a barcode.

FIG. 6 shows the structures of the vectors p1stBC, p2ndBC and p3rdBC used for incorporating barcodes.

FIG. 7 shows a method for preparing a vector to be used for incorporating a target gene.

FIG. 8 shows introduction of an URA3 lacO fragment into a 2-copy rDNA strain, and amplification of the URA3 lacO fragment.

FIG. 9 shows the results of pulse field electrophoresis analysis of URA3 lacO fragment amplification.

FIG. 10 shows results from fluorescent microscopy confirming that an URA3 lacO cassette was amplified together with rDNA.

FIG. 11 shows a systematic gene insertion system using the R-recombinase expression plasmid YEp181GALpR (GALp-Rec) as a vector for incorporating a target gene.

FIG. 12 illustrates the expression of a human RNAi-associated protein by an artificial chromosome vector.

FIG. 13 illustrates the reconstruction of a human siRNA production system in a yeast.

FIG. 14 illustrates gene silencing by human siRNA.

FIG. 15 shows a protocol for introducing the SIR2 gene upstream from the TRP1 gene on chromosome 4 of a budding yeast.

FIG. 16 shows PCR results confirming insertion of 15 barcode sequences.

### DESCRIPTION OF EMBODIMENTS

Embodiments of the present invention (hereunder called “the embodiments”) are explained below, but the scope of the invention should not be construed as being limited to the following embodiments.

### Artificial Chromosome Vector

The first embodiment provides an artificial chromosome vector derived from a eukaryotic chromosome, comprising repetitive units of multiple ribosomal RNA genes (rDNA) and intergenic regions between the repetitive units, wherein an intergenic region 1 (IGS1) of the intergenic regions comprises a barcode sequence for incorporating and distinguishing a target gene.

The eukaryote from which the artificial chromosome vector is derived is not limited to higher mammals such as humans and other mammals, and comprises eukaryotic plans such as liverworts, and fungi such as yeasts that retain the characteristics of higher eukaryotes, including unicellular organisms such as budding yeasts and fission yeasts for example. Of the eukaryotes, a yeast is preferred, and a budding yeast such as Saccharomyces cerevisiae is particularly desirable.

As used herein, an “artificial chromosome vector” is a vector that uses the replication mechanism of a eukaryotic chromosome, and which is capable of autonomous replication and distribution to daughter cells in host cells, can be retained stably in host cells, and can exist independently from the chromosomes of the host cells.

Ribosomes are the most abundant protein-RNA complexes in cells, and their genes (rDNA) also occur in multiple copies (rDNA repeats). The rDNA of a eukaryotic cell is a repetitive gene that is repeated 100 times or more on the chromosome. In budding yeasts for example, about 150 copies are tandemly repeated on chromosome 12, constituting about 10% of the total genome of the budding yeast. In the case of budding yeasts, one repetitive unit constitutes about 9.1 kb, of which about 6 kb is the gene (35S, 5S rDNA) and the remaining roughly 3 kb is a non-coding region included in an intergenic region.

Ribosomes are essential translation mechanisms in all living organisms, so mechanisms for maintaining multiple copies of rDNA are also essential. In fact, even if the rDNA of a budding yeast is artificially reduced to two copies, it is then restored to 150 copies. That is, two copies (18 kb) are elongated to 150 copies (1.4 Mb). This recovery of copy numbers by amplification is also observed in other organisms.

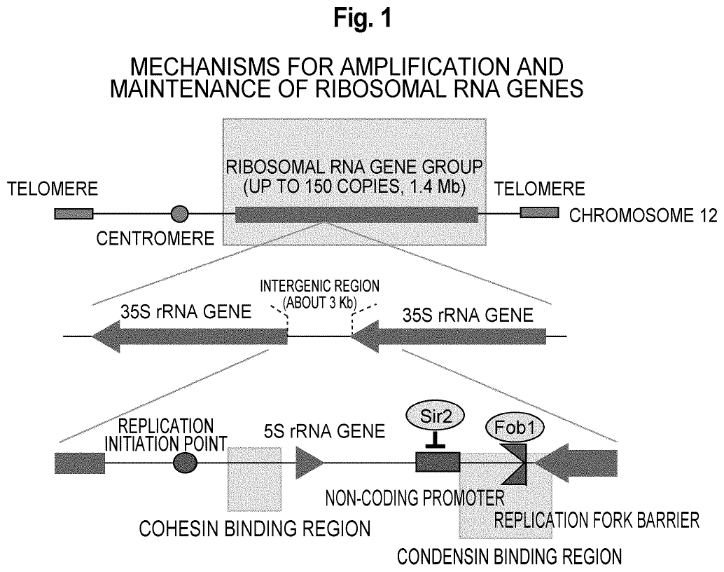

Eukaryotic rDNA is made up of coding regions and non-coding regions (also called nontranscription sequences (NTS)). Factors involved in maintaining rDNA chromosomes, such as cohesins involved in cohesion of sister chromatids and condensins involved in chromosome condensation, are concentrated between the rDNA genes, specifically in non-coding regions that do not code for proteins and are located in intergenic regions (intergenic spacers or IGS) (FIG. 1).

The intergenic spacers between repetitive units of rDNA are composed of two units called IGS1 and IGS2, with a 5S gene between the two. As used herein, an “intergenic spacer” is a region comprising an IGS1, a 5S rDNA sequence and an IGS2. Intergenic spacers are thought to be involved in regulating transcription and regulating transcription termination.

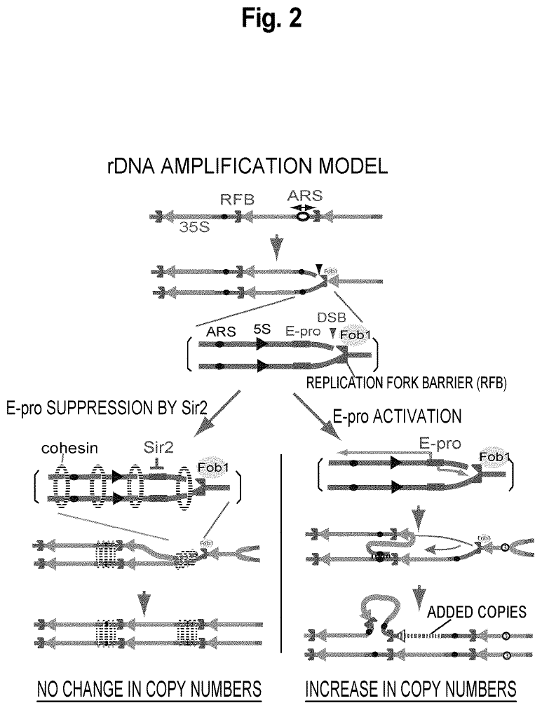

Unique functions of rDNA include gene amplification action to create multi-copy states and recombination suppression action to prevent copy loss. Our previous research has presented an amplification model that is dependent on DNA replication inhibition as shown in FIG. 2 (Kobayashi T, Ganley ARG (2005), Recombination regulation by transcription-induced cohesin dissociation in rDNA repeats, Science 309: 1581-1584; Kobayashi T, Horiuchi T, Tongaonkar P, Vu L, Nomura M (2004), SIR2 regulates recombination between different rDNA repeats, but not recombination within individual rRNA genes in yeast, Cell 117: 441-453).

In the model of FIG. 2, DNA double-strand break (DSB) occurs in replication fork barriers (RFB) located in the rDNA intergenic spacers, and the broken ends are repaired by reverting to homologous recombination with sister chromatids of adjacent copies. Because replication recommences at this point, the same region is replicated twice, and the number of copies increases (right of FIG. 2). It has been shown that this “revertive recombination repair” requires diversion of cohesin and condensin by transcription from non-coding promoters.

Regarding the other mechanism of copy number maintenance, the “recombination suppression action”, several mutant strains that reduce rDNA stability, or in other words with extreme copy number fluctuation, have already been identified.

An intergenic spacer contains an autonomously replicating sequence (ARS), a cohesin binding site, a 5S rDNA sequence, a non-coding promoter (E-pro) sequence and a replication fork barrier (RFB) sequence in that order from the 5′ side of the rDNA gene.

Constituent elements contained in the artificial chromosome vector comprise repetitive units of ribosomal RNA genes (rDNA) and intergenic spacers between the repetitive units. The number of repetitive units depends partly on the eukaryote from which the artificial chromosome is derived, but is about 2 to 150 in the case of a yeast. However, the upper limit need not be 150. When the yeast is a budding yeast, the repetitive units of rDNA are located on chromosome 12 of the budding yeast.

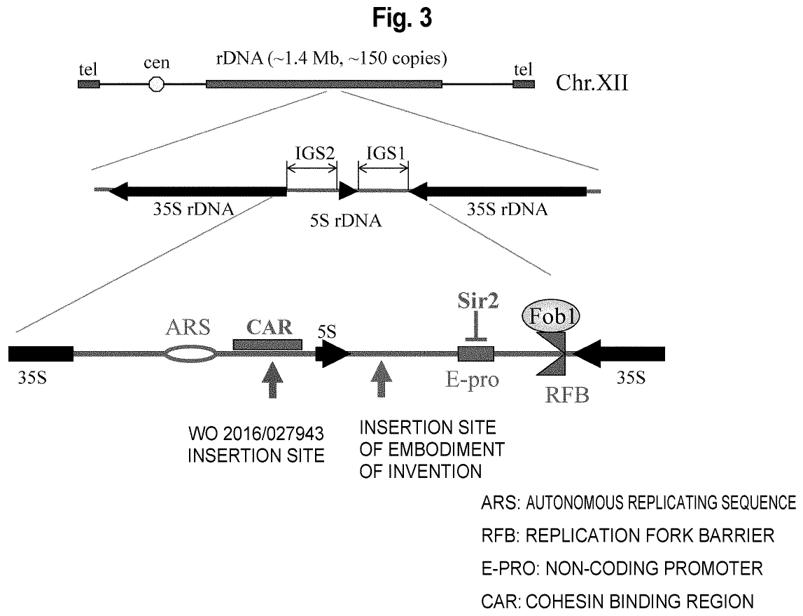

The artificial chromosome may also contain an autonomous replication sequence (ARS) to allow autonomous replication of the artificial chromosome vector, a cohesin binding site (CAR), a condensin binding site, and a telomere, centromere, non-coding promoter, replication fork barrier (RFB) and the like. As one example, the structure of budding yeast rDNA is shown in FIG. 3.

The structure shown in FIG. 3 is only an example, and the artificial chromosome vector may also contain a drug resistance gene, a selection marker gene for expression in the host (positive selection marker gene or negative selection marker gene) or the like. Examples of selection marker genes include genes coding for fluorescent proteins such as green fluorescent proteins (GFP or EGF), beta-galactosidase genes, luciferase genes, and genes involved in auxotrophy and the like. A selection marker used for insertion can be removed by homologous recombination or with a recombinase such as Cre-LoxP.

The intergenic region 1 (IGS1) comprises a barcode (BC) sequence for incorporating and distinguishing a target gene. As used herein, a “barcode sequence” is a nucleic acid for inserting a target gene into an artificial chromosome, and signifies a nucleic acid having a unique sequence for distinguishing among multiple target genes. Each barcode sequence has a sequence that is different from the others, or in other words a sequence unique to each rDNA copy, but depending on the objective a part of the barcode may have an identical sequence.

To prepare the barcode sequence, for example single-strand DNA having chemically synthesized random sequences can be treated with DNA polymerase to obtain double strands that are then tied together to prepare the barcode sequence. The barcode sequence is preferably one that does not easily form internal secondary structures (has no continuous GC sequences), and does not contain any sequences of restriction enzymes used for cloning. Examples of barcode sequences are shown in Table 4.

The barcode sequence insertion site may be any within the IGS1 that does not affect the desired effects, but is preferably between the 5S rDNA sequence and the non-coding promoter (E-pro) sequence, or between the E-pro sequence and the replication fork barrier (RFB).

From the standpoint of inserting the target gene, the barcode sequence preferably has a recognition site for a restriction enzyme or recombinase. Examples of such recognition sites include IoxP (Cre recombinase recognition site), FRT (Flp recombinase recognition site), φC31attB and φC31attP (φC31 recombinase recognition sites), R4attB and R4attP (R4 recombinase recognition sites), TP901-1attB and TP901-1attP (TP901-1 recombinase recognition sites), and Bxb1attB and Bxb1attP (Bxb1 recombinase recognition sites) and the like.

Enzymes for specifically causing recombination of a target gene or the like at these recognition sites include Cre integrase (Cre recombinase), Flp recombinase, φC31 integrase, R4 integrase, TP901-1 integrase, Bxb1 integrase and the like.

Each barcode sequence may also serve as a target for insertion of a target gene. The barcode sequence may also contain a promoter sequence or terminator sequence for controlling expression of the target gene. In this case, the terminator sequence may be arranged on the 3′ side, and the promoter sequence on the 5′ side of the target gene. The promoter may be any that is inducible in the host, without any special limitations, but examples of promoters that can be used in budding yeasts include inducible promoters such as galactose inducible (GAL) promoters, alkali phosphatase promoters (PHO), and the CUP1 promoter (which is activated by addition of copper ions), as well as constitutive promoters such as ADH1. Examples of GAL promoters include GAL1, GAL7 and GAL10.

The barcode sequence is preferably about 600 bp long because the efficiency of gene insertion by homologous recombination declines if the sequence is too short, while preparation becomes difficult if it is too long.

The barcode sequence can be inserted into the artificial chromosome

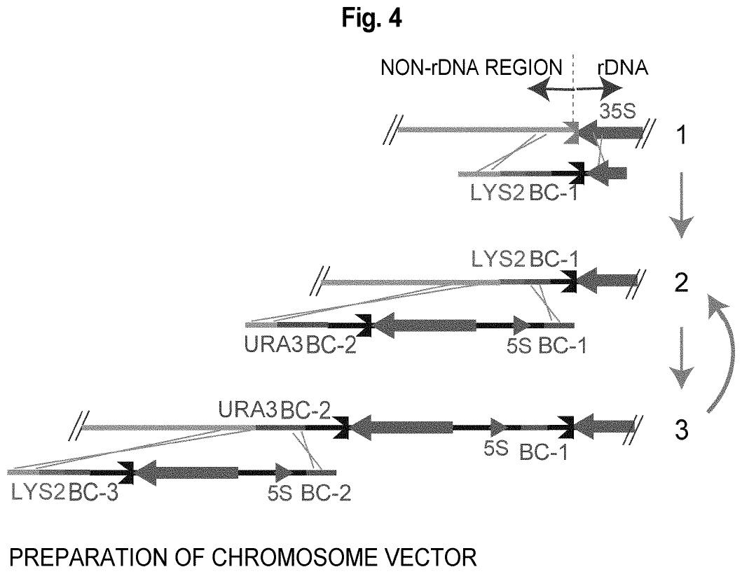

vector by a method known to those skilled in the art, such as homologous recombination or the like. For example, as shown in FIG. 4, 1) a non-rDNA sequence next to an rDNA repeat can be used to introduce a selection marker gene (LYS2) and a unique roughly 600 bp first barcode sequence (BC-1) into the vector by homologous recombination. Next, 2) BC-1 and a non-rDNA sequence can be used to introduce a new unique second barcode sequence (BC-2) adjacent to this together with a different selection marker gene (URA3) and rDNA, while at the same time removing the LYS2. 3) For the third barcode sequence, similarly a LYS2-BC-3-rDNA fragment is introduced by homologous recombination between BC2 and a non-rDNA sequence, and URA3 is removed at the same time. For the fourth and subsequent barcode sequences, rDNA copies having unique barcode sequences can be added successively by repeating steps 2) and 3) while changing only the barcode sequence, to ultimately construct the desired artificial chromosome vector.

Optional sequences such as adapter sequences for sequencing purposes can also be added to each barcode sequence.

rDNA repeats are also known to be formed and maintained on separate chromosomes (Oakes et al, Mol Cell Biol 26: 6223-6238, 2006). From the standpoint of efficiency, therefore, separate short chromosome vectors can be prepared in different cells, and these chromosomes can finally be combined by mating into one cell to quickly prepare a cell having multiple copies of the barcode.

From the standpoint of increasing rDNA stability, each barcode sequence is preferably inserted into a restriction enzyme site located between a 5S rDNA sequence and an E-pro sequence.

The target genes inserted into the barcode sequence can be determined appropriately by a person skilled in the art depending on the intended use of the artificial chromosome. The target genes may be homologous or heterologous to the host into which the artificial chromosome vector is introduced, and the genes may be the same or different.

The target genes may be placed under the control of suitable promoters, and may be incorporated into the artificial chromosome vector in combination with additional terminators, enhancers, markers and the like.

An artificial gene amplification system (in vivo PCR) capable of mass-producing useful proteins and the like can be achieved by preparing an artificial chromosome vector having identical genes inserted into multiple barcode sequences. Unlike conventional mass production systems using multi-copy plasmids, moreover, this system has the advantage of being able to stably maintain the amplified genes on the chromosome without the need for drug selection or the like.

Many physiological reactions involve multiple proteins forming protein complexes, and reconstruction of the reaction system required for analysis is not possible unless all factors are expressed. The problem with systems using plasmids is that the number of genes that can be inserted is limited, and plasmid stability (maintenance rates) may decline depending on the size and properties of the inserted genes. By contrast, because the artificial chromosome vector has a stability maintenance mechanism, it can introduce and maintain multiple heterologous genes very stably. In particular, the genes to be introduced can be accurately inserted one by one into the targeted copies by inserting barcode sequences unique to each rDNA copy. Consequently, by introducing into host cells an artificial chromosome vector coding for a human protein complex requiring analysis, it is possible to reconstruct the protein complex and its reaction system in different host cells.

Amplification of a target gene with an artificial chromosome vector can be controlled by turning on and off the Fob1 protein, which inhibits DNA replication and induces DNA double-strand break (FIG. 2). Fob1 binds specifically to a replication fork barrier (RFB) near the 35S rDNA transcription termination site, and acts to inhibit the progress of DNA replication forks from the autonomously replicating sequence (ARS). Fob1 also causes DNA double-strand break, and induces amplification recombination by unequal sister-chromatid recombination.

The RFB is involved in the rDNA stability maintenance mechanism in conjunction with the E-pro of a bidirectional non-coding promoter on rDNA. Expression of the E-pro is induced by a reduction in the number of rDNA copies, and increases the number of rDNA copies (FIG. 2, lower right). The sirtuin protein Sir2 controls the number of rDNA copies by controlling E-pro (FIG. 2, lower left).

### Eukaryotic Cell

The second embodiment provides a eukaryotic cell comprising the artificial chromosome vector.

Because the artificial chromosome vector is constituted with rDNA

intergenic spacers, a eukaryotic cell into which the artificial chromosome vector has been introduced can maintain a fixed number of copies of the artificial chromosome without disrupting the genes contained therein.

The eukaryotic cell comprising the artificial chromosome is not particularly limited, and examples include yeasts and other microbial cells. Various known species of yeasts may be used, and examples include Saccharomyces species such as Saccharomyces cerevisiae, Schizosaccharomyces species such as Schizosaccharomyces pombe, Candida species such as Candida shehatae, Pichiaspecies such as Pichia stipitis, Hansenula yeasts, Klocckera yeasts, Schwanniomyces yeasts, Yarrowia yeasts, Trichosporon yeasts, Brettanomycesyeasts, Pachysolen yeasts, Yamadazyma yeasts, Kluyveromyces species such as Kluyveromyces marxianus and Kluyveromyces lactis, and Issatchenkia species such as Issatchenkia orientalis and the like. From the standpoint of versatility, a Saccharomyces yeast is preferred. Of these a budding yeast is preferred, preferably Saccharomyces cerevisiae.

Methods for introducing the artificial chromosome vector into the eukaryotic cell include for example calcium phosphate methods, transformation methods, transfection methods, conjugation methods, protoplast methods, electroporation methods, lipofection methods and the like. The transformed eukaryotic cells can be selected using a marker such as target gene activity or expression of a marker gene.

### Method for Manufacturing Artificial Chromosome Vector

A third embodiment provides a method for manufacturing an artificial chromosome vector, comprising a step of inserting a barcode sequence into an IGS1.

The insertion site of the barcode sequence may be anywhere within the

IGS1 as long as the desired effects are not affected, but is preferably between an 5S rDNA sequence and a non-coding promoter (E-pro) sequence.

As explained previously with reference to FIG. 4, barcode sequence insertion can be achieved by methods known to those skilled in the art, such as homologous recombination. For example, multiple barcode sequences can each by inserted into a different IGS1 by following the steps:
1) A step of inserting a first barcode sequence and a selection marker sequence into a first IGS1 by homologous recombination of rDNA and a non-rDNA gene region;
2) A step of inserting a second barcode sequence and a selection marker sequence different from the selection marker sequence of 1) into a second IGS1 by homologous recombination of the first barcode sequence inserted in step 1) and a non-rDNA gene region.

As used herein, a “non-rDNA gene region” means a region present in an rDNA repeat that is not a ribosomal RNA gene (rDNA) sequence or an intergenic spacer.

Insertion of a third and subsequent barcode sequences can be achieved by further repeating the following step in sequence:
3) A step of inserting a third barcode sequence and a selection marker different from the selection marker of 2) into a third IGS1 by homologous recombination of the second barcode sequence inserted in step 2) and a non-rDNA gene region.

Homologous recombination may also be performed using a homologous recombination vector comprising each barcode sequence together with a selection marker sequence. To obtain the homologous recombination vector, a DNA cassette for insertion can be linked between sequences homologous to the bases of the 5′-end region and 3′-end region of the barcode sequence. Examples of homologous recombination vectors include plasmids, phages, cosmids and viruses, and a plasmid is preferred. The basic vector for constructing the homologous recombination vector may also comprise sequences commonly inserted during vector construction, such as a promoter, terminator, enhancer, selection marker gene, replication initiation point and the like.

The selection marker gene may be a uracil synthesis gene or lysine synthesis gene.

Although this is not intended as a limitation, one example of a method for manufacturing an artificial chromosome vector is explained below.

### Insertion of First Barcode Sequence

A plasmid (1^{st}BC) is prepared comprising a unique roughly 600 bp first barcode sequence (BC-1) and a selection marker gene (LYS2) sandwiched between a non-rDNA sequence and the region from the 35S rDNA 3′ sequence to IGS1, and the vector part is excised and introduced into a host having low-copy rDNA. When selected in medium containing no lysine, the 35Sr DNA and non-rDNA sequence are each exchanged by homologous recombination with the homologous regions of the host, and a strain having BC-1 and LYS2 introduced into the rDNA terminal region is isolated as a result.

### Insertion of Second Barcode Sequence

A plasmid (2^{nd}BC) is then prepared comprising a second barcode sequence (BC-2), a selection marker gene (URA3) and an rDNA1 copy having a hygromycin resistance mutation, sandwiched between BC-1 and a non-rDNA sequence, and the vector part is excised and introduced into the strain having BC-1 and LYS2 inserted in the rDNA terminal region. When selected in medium containing no uracil, the BC-1 and non-rDNA sequence are each exchanged by homologous recombination with the homologous regions of the host, with the result that the LYS2 is eliminated and a strain having BC-2, rDNA and URA3 introduced into the rDNA terminal region is isolated.

### Insertion of Third Barcode Sequence

A plasmid (3^{rd}BC) is then prepared comprising a third barcode sequence (BC-3), a selection marker gene (LYS2) and an rDNA1 copy having a hygromycin resistance mutation, sandwiched between BC-2 and a non-rDNA sequence, and the vector part is excised and introduced into the strain having BC-2 and URA3 inserted into the rDNA terminal region. When selected in medium containing no lysine, the BC-2 and non-rDNA sequence are each exchanged by homologous recombination with the homologous regions of the host, with the result that the URA3 is eliminated and a strain having BC-3, rDNA and LYS2 introduced into the rDNA terminal region is isolated.

The operations performed when inserting the second and third barcode sequences can be repeated with only the barcode sequence changed to construct a chromosome vector having added rDNA copies with unique barcode sequence in order from number one.

When the number of barcode sequence inserted into the chromosome vector is increased, the ribosomal RNA gene may be destabilized, and copies may be lost. When a large number of barcode sequences is inserted, therefore, such as when the number of barcode sequences is 10 or more for example, it is desirable to further stabilize the ribosomal RNA gene. For example, the sirtuin protein Sir2 controls the number of copies of ribosomal RNA genes, and it is known that ribosomal RNA genes can be stabilized by overexpressing the SIR2 gene coding for Sir2 (Ganley, A.R.D., Ide, S., Saka, K. and Kobayashi, T. (2009), The effect of replication initiation on gene amplification in the rDNA and its relationship to aging, Molecular Cell 35, 683-693). Ribosomal RNA genes are also stabilized by disrupting the EAF3 gene, which codes for one yeast NuA4 acetyltransferase complex subunit (Wakatsuki, T., Sasaki, M. and Kobayashi, T. (2019), Defects in the NuA4 acetyltransferase complex increase stability of the ribosomal RNA gene and extend replicative lifespan, Genes Genet. Systems 94, 197-206).

Consequently, when the number of barcode sequences contained in an artificial chromosome vector is large, such as 10 or more for example, the ribosomal RNA gene can be stabilized and copy loss can be prevented by either introducing the SIR2 gene into the host cells or disrupting the EAF3 gene in the host cells. The introduction site of the SIR2 gene is not particularly limited, but if the host is a budding yeast for example the SIR2 gene can be introduced upstream from the TRP1 gene on chromosome 4. Instead of introducing the SIR2 gene, the SIR2 gene can be cloned to a plasmid vector and used to overexpress the gene in the host cells. Disruption of the EAF3 gene can be achieved by common methods. For example, part or all of the EAF3 gene can be deleted, or another gene may be inserted in the middle or the like to eliminate the function of the gene.

### Uses for Artificial Chromosome Vector

A fourth embodiment provides various methods using the artificial chromosome.

For example, because the artificial chromosome vector having rDNA repeats has a unique barcode inserted for each rDNA copy, the target gene to be introduced can be accurately inserted into the target copies one by one. Therefore, multiple identical or different target genes or their expression products can be manufactured using the artificial chromosome vector.

Manufacture of a target gene or its expression product may include
1) A step of inserting a target gene into a barcode sequence of an artificial chromosome vector, and
2) A step of maintaining and culturing in host cells the resulting artificial chromosome vector obtained in 1).

When the host expresses a replication inhibiting protein such as Fob1 in the culture step, thereby inducing rDNA amplification, the inserted target gene is also amplified together with rDNA. Amplification can be confirmed by Southern analysis, quantitative PCR, pulse field electrophoresis or the like.

To manufacture large quantities of a target gene or its expression product, identical target genes are inserted into the artificial chromosome vector.

The host and the host culture conditions can be selected appropriately by a person skilled in the art depending on the object. To mass produce the target gene for example, it is desirable to increase the number of rDNA copies. In yeasts for example, it is known that rDNA is destabilized (fluctuation of rDNA copy numbers increases) and the number of copies increases to about 3 times the normal amount in strains lacking CTF4 (which is involved in DNA replication) and RTT109 (which is involved in recombination).

CTF4 is a constituent protein of the replisome. The replisome is composed of CMG helicase, which unwinds template DNA, DNA polymerase ε, which is responsible for leading strand synthesis, DNA polymerase α-primase complex, which initiates lagging strand synthesis, DNA polymerase ξ, which is responsible for lagging strand elongation, and accessory proteins.

When CTF4 is lacking, the ends of DNA double-strand breaks undergo resection and are repaired by the homologous recombination pathway, resulting in an abnormal increase in the number of rDNA copies.

RTT109 is a histone acetyltransferase, and its gene is associated with changes in chromatin structure. It is known that when RTT109 gene expression declines, rDNA begins rolling circle-type DNA replication, and the number of copies increases.

An rDNA unstable strain can also be prepared by other methods. For example, rDNA can be destabilized by expressing large quantities of Fob1, or deleting some of the DNA replication origin to weaken initiation activity. Destabilization can be evaluated by looking at differences between cells in the length of the chromosome 12 having the rDNA, which appears as a broad band (smear) in electrophoresis. The amount of circular molecules (circular rDNA) excised from rDNA by recombination is also reported to correlate to rDNA destabilization, and destabilization can therefore be confirmed by measuring this amount.

The expressed amount of the target gene or its expression product can be controlled by methods known to those skilled in the art. For example, when the host produces a protein or a gene for a protein such as Fob1 that inhibits replication of DNA and induces double-stranded DNA breakage, the expressed amount can be controlled by turning on and off the protein or gene.

An experimental system using the artificial chromosome vector allows physiological effects that do not show activity to be analyzed in an in vitro experimental system using purified proteins. For example, DNA replication and recombination reactions involve many factors and are difficult to reconstruct in vitro, and necessary and sufficient factors have yet to be determined. Even in such cases, a series of factors associated with human DNA replication reactions can be inserted into a yeast chromosome vector, and the human replication system can then be operated and analyzed in the yeast.

Although this is not intended as a limitation, the artificial chromosome vector can be used in the following analytical methods.
A gene associated with a human DNA repair system is introduced into the artificial chromosome vector, and the human repair system is reconstructed in a yeast to analyze the mechanisms of cancer development or cell aging.
Genes involved in research using mice or other individual animals are introduced into the artificial chromosome vector, and the genes or their functions are analyzed in a yeast or other host having the introduced vector.
Genes associated with a complex of multiple proteins that work together are introduced into the artificial chromosome vector, and reconstructed in a yeast to analyze their functions.
A host yeast is mutagenically treated with a mutant form of a gene introduced into an artificial chromosome vector, and the behavior is analyzed.
Genes from different species of organism are introduced into the artificial chromosome vector, and their functions are analyzed.
A gene associated with photosynthesis is introduced into the artificial chromosome vector, a yeast capable of using light energy is created, and the behavior is analyzed.
A gene associated with heterochromatin production is introduced into the artificial chromosome vector, and its behavior is analyzed.
Genes associated with human histone, transcription-related factors, translation-related factors and protein repair factors are introduced into the artificial chromosome vector, and a yeast having a human protein production system is prepared and used for drug discovery and analyzing drug targets.
Genes associated with disease and genes associated with metabolic systems in humans and useful organisms are introduced into the artificial chromosome vector, and used for drug discovery and analyzing drug targets.

This analysis can be performed for example by a method of analyzing the behavior of multiple different target genes derived from cells of the same or different species, or expression products of these target genes, in host cells, comprising:
1) A step of maintaining and culturing an artificial chromosome vector having the target genes inserted into barcode sequences in a host; and
2) A step of analyzing the functions of the target genes, or their expression products, or their behaviors in the host.

The above steps are only examples, and necessary steps may be included as appropriate either in step 1) or 2) or before or after these steps. For example, the expression level of an expression product may also be measured in step 2). For the expression level, either the amount of a transcription product of a target gene or the amount of a translation product may be measured.

Furthermore, step 2) may be implemented in the presence or absence of a substance that activates or inhibits the target gene or its expression product. Step 2) may also comprise a step of comparison with a host into which the artificial chromosome vector has not been introduced.

### Artificial Cell

A fifth embodiment provides an artificial cell comprising the artificial chromosome vector in an artificial cell membrane such as a liposome.

rDNA is basic genetic material found in all organisms, and plays a central role in metabolism. For example, the genes of the roughly 80 kinds of liposome proteins can all be cloned to an artificial chromosome vector built on such rDNA. An artificial cell can be created by combining such an artificial chromosome vector with liposomes.

An artificial cell can be given physiological activity by cloning genes coding for functional proteins to the artificial chromosome vector.

Examples of artificial cell membranes include liposomes and micelles. Liposomes are lipid bilayer vesicles, and can be prepared by known methods by a person skilled in the art. Micelles are preferably mixed micelles made up of lipids and surfactants.

The present invention is explained in more detail below based on examples, but the invention is not limited to these examples.

### EXAMPLES

### 1. Barcode Preparation

The barcode is prepared by first making synthesized single-stranded DNA oligonucleotides into double-stranded DNA fragments, ligating these fragments together, and then assembling the multiple DNA fragments by a polymerase chain reaction (PCR). The synthetic DNA oligonucleotides that were used are shown in Tables 1 to 3.

TABLE 1
Oligo-DNA

| Oligonucleotide | |
|---|---|
| name | Sequence |
| OLI001 | ACACTCTTTCCCTACACGACGCTGT |
| | TCCGATCTnnnnnnnnnnnnnnnnn |
| | nnnnnnnnAGATCGGAAGAGCACAC |
| | GTCTG (SEQ ID NO: 1) |
| OLI002 | 5′ phospho-ATCnnnnnnnnnnn |
| | nnnnnnnnnnnnnnnnnnnnnnnnn |
| | nnnnnnnnnnnnnnnnnnnnnnnnn |
| | nnnnnnnnnnnnnnnCAGACGTGTG |
| | CTCTTCCGATCT (SEQ ID NO: |
| | 2) |
| OLI003 | AGATCGGAAGAGCACACGTCTG |
| | (SEQ ID NO: 3) |
| OLI004 | 5′ phospho-GGGnnnnnnnnnnn |
| | nnnnnnnnnnnnnnnnnnnnnnnnn |
| | nnnnnnnnnnnnnnnnnnnnnnnnn |
| | nnnnnnnnnnnnnnnnnnnGCAATC |
| | TACAAGG (SEQ ID NO: 4) |
| OLI006 | CCTGTAGATAGATTGC (SEQ ID |
| | NO: 5) |
| OLI006 | CTGnnnnnnnnnnnnnnnnnnnnnn |
| | nnnnnnnnnnnnnnnnnnnnnnnnn |
| | nnnnnnnnnnnnnnnnnnnnnnnnn |
| | nnnnnnnnCCTTGTAGATAGATTGC |
| | (SEQ ID NO: 6) |
| OLI007 | GCAATCTATCTACAAGG (SEQ ID |
| | NO: 7) |
| OLI008 | 5′ phospho-ATCnnnnnnnnnnn |
| | nnnnnnnnnnnnnnnnnnnnnnnnn |
| | nnnnnnnnnnnnnnnnnnnnnnnnn |
| | nnnnnnnnnnnnnnnnnnnCGTAAG |
| | TTCTATGCTTG |
| | (SEQ ID NO: 8) |
| OLI009 | CAAGCATAGAACTTACG (SEQ ID |
| | NO: 9) |
| OLI010 | 5′ phospho-CTGnnnnnnnnnnn |
| | nnnnnnnnnnnnnnnnnnnnnnnnn |
| | nnnnnnnnnnnnnnnnnnnnnnnnn |
| | nnnnnnnnnnnnnnnnnnnCAAGCA |
| | TAGAACTTACG |
| | (SEQ ID NO: 10) |
| OLI011 | CGTAAGTTCTATGCTTGCGTAAGTT |
| | CTATGCTTG |
| | (SEQ ID NO: 11) |
| OLI012 | 5′ phospho-ATCnnnnnnnnnnn |
| | nnnnnnnnnnnnnnnnnnnnnnnnn |
| | nnnnnnnnnnnnnnnnnnnnnnnnn |
| | nnnnnnnnnnnnnnnnnnnGTACGA |
| | ACGCTTCAGACC |
| | (SEQ ID NO: 12) |
| OLI013 | GGTCTGAAGCGTTCGTAC |
| | (SEQ ID NO: 13) |
| OLI014 | ACACTCTTTCCCTACACGACGCTC |
| | (SEQ ID NO: 14) |
| OLI015 | GTACGAACGCTTCAGACCAGCGCTG |
| | Caggcgtaatcatggtcatagctg |
| | (SEQ ID NO: 15) |
| OLI016 | GAGCGTCGTGTAGGGAAAGAGTGTA |
| | GATCTactggccgtcgttttacaac |
| | (SEQ ID NO: 16) |
| OLI017 | GTCGACagcgctCTGCAGgcgtaat |
| | catggtcatagctgtttCCTGTGAA |
| | ATTGTTATCCGCTCAC |
| | (SEQ ID NO: 17) |
| OLI020 | ATTACCCCGccctttcgtctcgcgG |
| | AAAATATCCATTACCCACAGTAAGA |
| | C (SEQ ID NO: 18) |
| OLI021 | gcCTGCAGagcgctGTCGACgtatc |
| | acgaggccagcttttcaagaggcat |
| | cgcacagttttagcgagg |
| | (SEQ ID NO: 19) |
| OLI022 | cgcgagacgaaagggCGGGTAATat |
| | catacgtaatgctcaaccTTAAGCT |
| | GC (SEQ ID NO: 20) |
| OLI023 | CCATATGCCAAATTGAGGGAcCCAA |
| | ATGTTATTTC(SEQ ID NO: 21) |
| OLI024 | GAAATAACATTTGGgTCCCTCAATT |
| | TGGCATATGG |
| | (SEQ ID NO: 22) |
| OLI025 | gtaaaacgacggccaCCTAGGTATA |
| | TATAACCATCAAGTAATACATATTT |
| | AACTTAGC |
| | (SEQ ID NO: 23) |
| 0LI026 | CCTAGGtggccgtcgttttacaacG |
| | TCGTGACTGGGAAAACCCTGGCGTT |
| | A (SEQ ID NO: 24) |
| OLI027 | gttgtaaaacgacggccaCCTAGGA |
| | GCGCTgcactcttcttctgaagagt |
| | taagc |
| | (SEQ ID NO: 25) |

TABLE 2

| | |
|---|---|
| OLI028 | agatctgcctagaagcagaattcgg |
| | taagcgttgg |
| | (SEQ ID NO: 26) |
| OLI029 | cgcCTGCAGagcgctGTCGACGGAT |
| | CCagtgcgaatttttctgaatcgaa |
| | tggtagg |
| | (SEQ ID NO: 27) |
| OLI030 | gaattctgcttctaggcagatctga |
| | cgatcacc (SEQ ID NO: 28) |
| OLI033 | CTGCAGagcgctGTCGACgtatcac |
| | gaggccagcttttc |
| | (SEQ ID NO: 29) |
| OLI034 | cgcgagacgaaagggCGGGTAATaa |
| | ctg (SEQ ID NO: 30) |
| OLI035 | cgcgagacgaaagggCGGGTAAT |
| | (SEQ ID NO: 31) |
| OLI036 | CCAGTGAGCCCGCTTGCAGAGTTC |
| | (SEQ ID NO: 32) |
| OLI037 | cattttggtagccaagatagaatg |
| | (SEQ ID NO: 33) |
| OLI038 | catatttgagtacgatcgttcctg |
| | (SEQ ID NO: 34) |
| OLI039 | caacccacgtggtttagcaacacc |
| | (SEQ ID NO: 35) |
| OLI040 | gctggcctcgtgatacGTCGACACA |
| | CTCTTTCCCTACACGACGC |
| | (SEQ ID NO: 36) |
| OLI041 | gcttaactcttcagaagaagagtgc |
| | AGCgctGGTCTGAAGCGTTCGTAC |
| | (SEQ ID NO: 37) |
| OLI042 | agcGCTgcactcttcttctgaagag |
| | ttaagc (SEQ ID NO: 38) |
| OLI043 | Gaaacagctatgaccatgattacgc |
| | CTGCAGTTCAATTCGGATAATTGG |
| | (SEQ ID NO: 39) |
| OLI044 | cgattcagaaaaatttcgcactGGA |
| | TCCACACTCTTTCCCTACACCACGC |
| | (SEQ ID NO: 40) |
| OLI045 | Gaaacagctatgaccatgattacgc |
| | CTGCAGGTCTGAAGCGTTCGTAC |
| | (SEQ ID NO: 41) |
| OLI059 | TCTTTCCCTACACGACGCTCGCGAT |
| | CGcggattagaagccgccgagcggg |
| | (SEQ ID NO: 42) |
| OLI060 | CTTTGTACAAGAAAGCTGGGTCatt |
| | aggcctGAAGCCTgcttttttgtac |
| | (SEQ ID NO: 43) |
| OLI061 | gtacaaaaaagcAGGCTTCaggcct |
| | aatGACCCAGCTTTCTTGTACAAAG |
| | (SEQ ID NO: 44) |
| OLI062 | gcaattaaagccttcgagcgtccc |
| | (SEQ ID NO: 45) |
| OLI063 | cgctcgaaaggctttaatttgcAAA |
| | GAAtacgttaTTCTTTCATCAAcca |
| | gcttttcaattcaattcatc |
| | (SEQ ID NO: 46) |
| OLI064 | GTCTGAAGCGTTCGTACGCGGCCGC |
| | AAAGAAtaacgtaTTCTTTCATCAA |
| | gctctaatttgtgagtttag (SEQ |
| | ID NO: 47) |
| OLI065 | GTAAAACGACGGCCAGTGaattCAC |
| | ACACCATAGGTTCAAAatgtttc |
| | (SEQ ID NO: 48) |
| OLI066 | TTTGTAATTAAAACTTAGATTAGat |
| | tgctatgc (SEQ ID NO: 49) |
| OLI067 | CTAAGTTTTAATTACAAACTTTTCG |
| | TTTGGATCTTTGGATAAGGCAGATT |
| | G (SEQ ID NO: 50) |
| OLI068 | cgttctcttttcaataaatttagtg |
| | ataaaatattcACATACctaatgcT |
| | CTAAAGGTGAAGAATTATTC (SEQ |
| | ID NO: 51) |
| OLI069 | aatttattgaaaagagaacgtTACT |
| | AACagtggaatttttttttcTAGaT |
| | CTAAAGGTGAAGAATTATTC (SEQ |
| | ID NO: 52) |
| OLI070 | ctattaaaaaaaataaataggCTTT |
| | TCGTTTGGATCTTTGGATAAGGCAG |
| | (SEQ ID NO: 53) |
| OLI071 | cctatttattttttttaatagttat |
| | gttag (SEQ ID NO: 54) |
| OLI072 | gaccatgattacgccaagctgcaga |
| | acctttagaggtaataagagataaa |
| | tac (SEQ ID NO: 55) |
| OLI073 | gtaaaacgacggccagtgaattcgt |
| | attctgataaatctaaagagaaatt |
| | ac (SEQ ID NO: 56) |
| OLI074 | AACTTTTCTTTTTTTTTTTGGTGGC |
| | ATtggcacaaatgtggtttcctttc |
| | ttattgc (SEQ ID NO: 57) |
| OLI075 | ATGCCACCAAAAAAAAAAAGAAAAG |
| | TTTCTAAAGGTGAAGAATTATTCAC |
| | TGG (SEQ ID NO: 58) |

TABLE 3

| | |
|---|---|
| OLI076 | gaccatgattacgccaagcttgccg |
| | gtagaggtgtggtcaataagagcg |
| | (SEQ ID NO: 59) |
| OLI082 | ATGAGCTGCAGGCCTGCCATGCTGA |
| | GGGGTTGCAAAGCAGGGCTTTTCAT |
| | GAAGCCTgcttttttgtacA (SEQ |
| | ID NO: 60) |
| OLI083 | GAAGAGCCCTCCGAAGCCTCAAAGC |
| | TAATCAACCTCAGGTTCCCAATAGC |
| | tGACCCAGCTTTCTTGTACA (SEQ |
| | ID NO: 61) |
| OLI084 | GGGGGGGGCGGCGCAGGAGGTGCAA |
| | GTGCGGGGCCGGCTCCCGAGTACAT |
| | GAAGCCTgcttttttgtacA (SEQ |
| | ID NO: 62) |
| OLI085 | CCAAGGCGGTCCAGGTTCACCAAGA |
| | CACTCTGCGCACCaTGTACTTTGCT |
| | tGACCCAGCTTTCTTGTACA (SEQ |
| | ID NO: 63) |
| OLI086 | CGCAGCCCGCAGCCCGTGGTAGTGC |
| | CGGAGCCTTGCTCCTCTTCACTCAT |
| | GAAGCCTgcttttttgtacA (SEQ |
| | ID NO: 64) |
| OLI087 | AGGCTGCCCGCCGTGCCCTGCAGTA |
| | CCTCAAGATCATGGCAGGCAGCAAG |
| | tGACCCAGCTTTCTTGTACA (SEQ |
| | ID NO: 65) |
| OLI1126 | AGAGTTGCCCCAGAAGAACA (SEQ |
| | ID NO: 66) |
| OLI1127 | GGCTTGGATGGAAACGTAGA (SEQ |
| | ID NO: 67) |
| pPCRsplit | TCCAAAGATCCAAACGAAAAGAGAG |
| GFPFw | A (SEQ ID NO: 68) |
| pPCRsplit | GCGCCTTATTTGTACAATTCATCCA |
| GFPRv | TA (SEQ ID NO: 69) |
| siGFP01 | TCTAAAGGTGAAGAATTATTCACTG |
| | GTGTTGTCCCAATTTTGGTTGAATT |
| | aGATGGTGAT |
| | (SEQ ID NO: 70) |
| siGFP02 | AGATGGTGATGTTAATGGTGACAAA |
| | TTTTCTGTCTCCGGTGAAGGTGAAG |
| | GTGATGCTAC |
| | (SEQ ID NO: 71) |
| siGFP03 | GTGATGCTACTTACGGTAAATTGAC |
| | CTTAAAATTTATTTGTACTACTGGT |
| | AAATTGCCAG |
| | (SEQ ID NO: 72) |
| siGFP04 | AAATTGCCAGTTCCATGGCCAACCT |
| | TAGTCACTACTTTAACTTATGGTGT |
| | TCAATGTTTT |
| | (SEQ ID NO: 73) |
| siGFP05 | TCAATGTTTTTCTAGATACCCAGAT |
| | CATATGAAACAACATGACTTTTTCA |
| | AGTCTGCCAT |
| | (SEQ ID NO: 74) |
| siGFP06 | AGTCTGCCATGCCAGAAGGTTATGT |
| | TCAAGAAAGAACTATTTTTTTCAAA |
| | GATGACGGTA |
| | (SEQ ID NO: 75) |
| siGFP07 | GATGACGGTAACTACAAGACCAGAG |
| | CTGAAGTCAAGTTTGAAGGTGATAC |
| | CTTAGTTAAT |
| | (SEQ ID NO: 76) |
| SiGFP08 | CTTAGTTAATAGAATCGAATTAAAA |
| | GGTATTGATTTTAAAGAAGATGGTA |
| | ACATTTTAGG |
| | (SEQ ID NO: 77) |
| siGFP09 | ACATTTTAGGTCACAAATTGGAATA |
| | CAACTATAACTCTCACAATGTTTAC |
| | ATCATGGCTG |
| | (SEQ ID NO: 78) |
| siGFP10 | ATCATGGCTGACAAACAAAAGAATG |
| | GTATCAAAGTTAACTTCAAAATTAG |
| | ACACAACATT |
| | (SEQ ID NO: 79) |
| siGFP11 | ACACAACATTGAAGATGGTTCTGTT |
| | CAATTAGCTGACCATTATCAACAAA |
| | ATACTCCAAT |
| | (SEQ ID NO: 80) |
| siGFP12 | ATACTCCAATTGGTGATGGTCCAGT |
| | CTTGTTACCAGACAACCATTACTTA |
| | TCCACTCAAT |
| | (SEQ ID NO: 81) |
| siGFP13 | TTACCAGACAACCATTACTTATCCA |
| | CTCAATCTGCCTTATCCAAAGATCC |
| | AAACGAAAAG |
| | (SEQ ID NO: 82) |

PCR was performed in a reaction system using a C1000 Touch thermal cycler (Bio-Rad: 1851148JA) and a KOD FX neo DNA polymerase kit (Toyobo: KFX 201), with a concentration of 1 μM of each oligonucleotide primer. Agarose electrophoresis to isolate the PCR products and DNA fragments was performed with a Mupid-exU electrophoresis machine (Mupid: EXU-1) using 0.5% to 2% agarose gel (Star Agarose: RIKAKEN, RSV-AGRP-500g) prepared with 1×TAE (40 mM Tris, 20 mM acetic acid, 1 mM EDTA) or 1×TBE (89 mM Tris, 89 mM boric acid, 2.5 mM EDTA) electrophoresis buffer.

The DNA fragments were purified from the agarose gel using a NucleoSpin Gel and PCR Clean-up kit (Takara: U0609C). Plasmid preparation and amplification were performed on E. coli JM109 (e14-(mcrA-), recA1, supE44, endA1, hsdR17(rₖ⁻, mₖ⁺), gyrA96, relA1, thi-1, D(lac-proAB), F′[traD36, proAB, lacI^{q}, lacZDM15]), and the plasmids were purified with a NucleoSpin Plasmid Quick Pure kit (Takara: U0615B).

Selection and culturing of E. coli transformants was performed in LB medium containing 100 mg/L of ampicillin sodium (Wako: 014-23302). The LB medium contained 1% tryptone (BD Bacto Tryptone: 211705), 0.5% yeast extract (BD Bacto Yeast Extract: 212750) and 1% sodium chloride (Wako: 191-01665), and the plate medium (agar medium) also contained 2% agar (BD Bacto Agar: 214010).

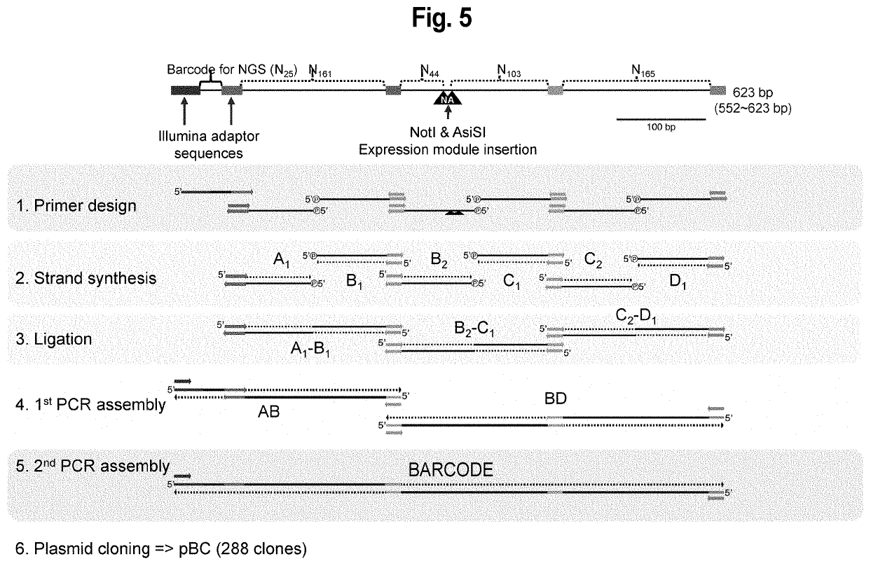

To double-strand the single-stranded DNA oligonucleotides, 17- to 22-nucleotide DNA oligonucleotides with complementary sequences were used as primers with KOD FX neo DNA polymerase to synthesize the complement strands for roughly 100-nucleotide single-stranded DNA oligonucleotides comprising random sequences (FIG. 5: 2. Strand synthesis). DNA synthesis was performed by preparing reaction solutions containing 3 μM each of OLI002 and OLI003 (A₁ fragment), OLI004 and OLI005 (B₁ fragment), OLI006 and OLI007 (B₂ fragment), OLI008 and OLI009 (C₁ fragment), OLI010 and OLI011 (C₂ fragment) and OLI012 and OLI013 (D₁ fragment), and reacting these by incubating for 2 minutes at 94° C., 30 seconds at 98° C., 30 seconds at 48° C. and 10 minutes at 68° C. Following the double-stranding reaction, the residual single-stranded DNA was broken down by adding 0.2 u/μl of Exonuclease I (GE Health Care: E70073Z) to the reaction solution and incubating for 15 minutes at 37° C.

The synthesized A₁, B₁, B₂, C₁, C₂ and D₁ fragments were isolated and purified by 1×TBE 2% agarose gel electrophoresis, and 50 ng portions of solution DNA obtained by mixing A₁ with B₁, B₂ with C₁ and C₂ with D₁ were each prepared to a final volume of 2 μl. Ligation of the DNA fragments was performed by first mixing 2 μl of the DNA fragment mixture with 1.8 μl of the 2×Quick Ligation reaction buffer of a Quick ligation kit (New England Biolabs: M2200S) and 0.2 μl of Quick T4 DNA ligase, and then letting this sit at room temperature for 2 hours (FIG. 5: 3. Ligation). For each ligated DNA fragment, a PCR reaction solution was prepared containing each oligonucleotide DNA primer set with OLI002 and OLI005 (A₁-B₁ fragment), OLI007 and OLI009 (B₂-C₁ fragment) or OLI011 and OLI013 (C₂-D₁ fragment) as a ligation reaction solution with a concentration of 0.02?0.04 μl/μl, and amplified by reacting for 2 minutes at 94° C., followed by 10 cycles of [10 seconds at 98° C., 15 seconds at 48° C., 30 seconds at 68° C.].

The amplified A₁-B₁, B₂-C₁ and C₂-D₁ fragments were each separated and purified by 1×TBE 2% agarose gel electrophoresis. An A₁-B₁ fragment (AB fragment) with an added NGS barcode sequence was amplified by performing a PCR reaction of 2 minutes at 94° C. followed by 9 cycles of [5 seconds at 98° C., 15 seconds at 48° C., 45 seconds at 68° C.] with the oligo-DNA primers OLI005 and OLI014 and 0.5 μM of OLI001 with the A₁-B₁ fragment adjusted to 1 ng/μl (FIG. 5: 4. 1^{st} PCR assembly, left).

An assembly of the B2-C1 and C2-D1 fragments (BD fragment) was obtained by adding the B2-C1 and C2-D1 fragments to a concentration of 1 ng/μl of each to a PCR reaction solution containing the oligo-DNA primers OLI007 and OLI013, and performing a PCR reaction of 2 minutes at 94° C. followed by 9 cycles of [5 seconds at 98° C., 15 seconds at 48° C., 45 seconds at 68° C.] (FIG. 5: 4. 1^{st} PCR assembly, right).

The amplified AB fragment and BD fragment were each separated and purified by 1×TBE 2% agarose gel electrophoresis. An assembly of the AB fragment and BD fragment (barcode fragment) was obtained by adding the AB and BD fragments to a concentration of 2 ng/μl of each to a PCR reaction solution containing the oligo-DNA primers OLI013 and OLI014, and performing a PCR reaction of 2 minutes at 94° C. followed by 7 cycles of [5 seconds at 98° C., 15 seconds at 48° C., 1 minute at 68° C.] (FIG. 5: 5. 2^{nd} PCR assembly).

The resulting barcode fragment was separated and purified by 1×TBE 1.5% agarose gel electrophoresis. The vector fragment (pUC-vector fragment) used for cloning the barcode fragment was obtained by using a pUC19 plasmid (Gene 1985, 33, 103-) as a template to prepare a PCR reaction solution containing the oligo-DNA primers OLI015 and OLI016, then amplifying by a reaction of 2 minutes at 94° C. followed by 30 cycles of [20 seconds at 95° C., 20 seconds at 48° C., 3 minutes 30 seconds at 68° C.], and separating and purifying by 1×TBE 2% agarose gel electrophoresis.

Assembly of the barcode fragment and vector fragment was performed by mixing 5 μl of a solution containing 0.13 pmol of the barcode fragment and 0.03 pmol of the pUC vector fragment with 5 μl of NEBuilder Master Mix solution (NEBuilder HiFi DNA Assembly Master Mix, NEW England BioLabs: E2621S), and then incubating this at 50° C. for 1 hour to perform a reaction. 2.5 μl of the assembly reaction solution was mixed with 50 μl of E. Coli JM109 competent cells, then coated on LB ampicillin plate medium and cultured overnight at 37° C. to transform the cells.

For the barcode plasmid (pBC), colonies grown at 37° C. were cultured in LB ampicillin liquid medium and purified with a plasmid purification kit, and the barcode sequence was determined by the Sanger sequence method (FIG. 5: 6. Plasmid cloning). Of the resulting pBC clones, one having a common structure with the sequence of the barcode region, one having no sequence of 6 or more continuous cytosines (C) or guanines (G), and one having no recognition sequence for the restriction enzymes AvrII, BamHI, Eco47III and PstI were selected and as barcodes. The barcodes used in the examples are shown in Table 4, and the barcode sequences comprising these are called pBC1, pBC2 and pBC3, respectively.

TABLE 4

| Sequence name | Barcode DNA sequence |
|---|---|
| BC1 | AGATCTacactctttccctacacga |
| | cgctcttccgatctGTTCGTGCCTT |
| | AGTTGTTTATGGTGagatcggaaga |
| | gcacacgtctgTAAGCAAGCTGCGA |
| | CAGGAAAGATCCCTCTTACCAAAAG |
| | ACAACTCGGCGAACTTCGGCTGGGG |
| | TCCCTAGTTCTTCCCTTGGCCGGTT |
| | TTTACGGGGATAGCCTCGGCTCACT |
| | GCTTATCATTCGTCTATTTCCTAAT |
| | TGCgcaatctatctacaaggGTTTC |
| | CGCTAATTGAGACTATAGGATGTTT |
| | CGCGCCATATGGTCgcggccgcAAg |
| | cgatcgcCAACAAAATCTAATTACC |
| | CAGATCCTTGTTCCCGTTGAATCTC |
| | GGTTACGCCCGGACGCGTCCGCATC |
| | AGGTCGCTTATCCGGATTGCAGTTA |
| | ATGAGCTAAAcgtaagttctatgct |
| | tgACTCTTACCATAGTGCACAGGGC |
| | GCTACCGAAAAAAATCGAACGGCGG |
| | GCGTAATCAGAACGCTACGGCGGGG |
| | TATATTCCAGATCCTATTGGAGGAA |
| | TGTGCTGTCGGTGTGATGTAGTGGA |
| | GCCGAATATGTAGGTTATCTGTTTG |
| | TCTGTCTGGTTGTGGGCgtacgaac |
| | gcttcagaccAGCGCTGCA (SEQ |
| | ID NO: 83) |
| BC2 | AGATCTacactctttccctacacga |
| | cgctcttccgatctCTTTTTCGTAT |
| | GTTGGTGCCTATTTagatcggaaga |
| | gcacacgtctgTCGGCCCGGACGAC |
| | GGCTGCACCAAAAAGAACCCAGGAC |
| | CCCACATCCGAAGCTTTCCAGAGCA |
| | GGCATCAAGCCGATGCGTGTGGGTC |
| | TTGCTCTTGTGTTATTTCGTGCCGG |
| | GGTCTGTTTTGTGGTCGGGTGATGA |
| | GCTCAgcaatctatctacaaggACC |
| | ATACGGAAACCAAGACTTTTGATCA |
| | GACCGGGGCCGACGTTgcggccgcA |
| | AgcgatcgcTTATCCACCATAATCA |
| | GACAGATCCAGGTGCCGTGTCACGT |
| | TACAGGTTTCGGGTAGGAACTGGCC |
| | TGTTGTCCGTGTAGGCCTGTTACAT |
| | TCACAGCACATCcgtaagttctatg |
| | cttgACATTAGCGCCCTAATTAAGA |
| | CACAACTACATTCCAATCAGCATTG |
| | CAGCGGAACACGAAAGGGCCTAGCC |
| | AATTAGGAGCAGTCGTATCATTTGG |
| | TCTGGGGTGGTAACTTGGGTTGAGC |
| | TTATTTTGTGTTGAGACTGTAGCGG |
| | GTGGTAGCATGGGATGATgtacgaa |
| | cgcttcagaccAGCGCTGCA (SEQ |
| | ID NO: 84) |
| BC3 | AGATCTacactctttccctacacga |
| | cgctcttccgatctTCTTAGTGGTC |
| | TTTCCACTCTTTGGagatcggaaga |
| | gcacacgtctgCCTGCCACAAACTG |
| | ACGTAACATAAAGCCATATACGCTC |
| | CAGATGCATAGAGGCGCGCTCCCAG |
| | AAAAATAGCAGGATGGGGACTGTGG |
| | GTTTGGGAAGTGGATATCCTCGCTA |
| | GGTCGTCTCGGTCCCGTCTCAGATT |
| | TAGCGTATGTACCATGATGTTTgca |
| | atctatctacaaggATAATAAACGT |
| | TATCCCCTAATACGTTGACGGAGAT |
| | GGACCCCAgcggccgcAAgcgatcg |
| | cCAATCAACAGGTAGAAACCATCGG |
| | CGTTTATCTTGGGTTGCAGTGTGTT |
| | TTCGCTCGCTTGTTGGCTTGTCAGG |
| | GTGTGCGGACTCCAACAGTCTCCAA |
| | TAcgtaagttctatgcttgTATCCG |
| | TGTGGCCCGAGAGCCGAAAATGTCG |
| | ACCACTATCCACACAGCCCTCACAC |
| | AAGTTCAGAACAGATCTCATCCTTG |
| | AGATTCCAGACTAAGGAGTGTAATC |
| | GCGGTATGCTATTTGGCAAATCTGT |
| | CCCATAATTGGCCTGGAACCgtacg |
| | aacgcttcagaccAGCGCTGCA |
| | (SEQ ID NO: 85) |

To prepare vectors for incorporating the barcodes, a plasmid pUC-_RDN1L_URA3 and a plasmid pUC_RDN1L_LYS2 were constructed each having the sequence RDN1L (SGD, S288C genome chrXII: 450760 . . . 451418) near the ribosomal RNA gene and the auxotrophic marker gene URA3 or LYS2. Plasmids pUCEco47RDNheadBamHI and pUCEco47RDNhyg1BamHI were also prepared and used as plasmids for contributing hygromycin B resistance ribosomal RNA genes (rDNA).

The backbone (vector fragment) of the plasmid was amplified by PCR for 2 minutes at 94° C. followed by 33 cycles of [20 seconds at 95° C., 20 seconds at 48° C., 3 minutes 30 seconds at 68° C.] using the OLI017 and OLI026 oligo-DNA primers and the template plasmid pUC19, and then separated and purified by agarose gel electrophoresis. The URA3 fragment was amplified by PCR for 2 minutes at 94° C. followed by 33 cycles of [20 seconds at 95° C., 20 seconds at 48° C., 1 minute 30 seconds at 68° C.] using the OLI033 and OLI034 oligo-DNA primers and the template plasmid YIplac211 (Gene 1988, 74, 527-), and then separated and purified by agarose gel electrophoresis.

The RDN1L fragment, LYS2-1 fragment, rDNA_SmaI-BamHI fragment and rDNA_NheI-Eco47III fragment were amplified by PCR for 2 minutes at 94° C. followed by 33 cycles of [20 seconds at 95° C., 20 seconds at 48° C., 1 minute 30 seconds at 68° C.] using genome DNA templates purified from the budding yeast strain BY4743 (Yeast 1988, 14, 115-) with the corresponding oligo-DNA sets OLI020 and OLI025, OLI022 and OLI023, OLI029 and OLI030, and OLI027 and OLI028, and separated and purified by agarose gel electrophoresis.

The LYS2-2 fragment was amplified by PCR for 2 minutes at 94° C. followed by 33 cycles of [20 seconds at 95° C., 20 seconds at 48° C., 3 minutes 30 seconds at 68° C.] using a genome DNA template purified from the budding yeast strain BY4743 (Yeast 1988, 14, 115-) with the oligo-DNA set OLI021 and OLI024, and separated and purified by agarose gel electrophoresis. 0.05 pmol of the vector fragment, 0.1 pmol of the RDN1L fragment and 0.1 pmol of the URA3 fragment (total 2.5 μl) were mixed with 2.5 μl of NEBuilder Master Mix to obtain a pUC_RDN1L_URA3 assembly reaction solution. 0.05 pmol of the vector fragment, 0.1 pmol of the RDN1L fragment, 0.1 pmol of the LYS2-1 fragment and 0.1 pmol of the LYS2-2 fragment (total 2.5 μl) were also mixed with 2.5 μl of NEBuilder Master Mix to obtain a pUC_RDN1L_LYS2 assembly reaction solution. For the pUCEco47RDNheadBamHI assembly reaction, 0.05 pmol of the vector fragment, 0.1 pmol of the rDNA_SmaI-BamHI fragment and 0.1 pmol of the rDNA_NheI-Eco47III fragment (total 2.5 μl) were mixed with 2.5 μl of NEBuilder Master Mix.

Each assembly reaction solution was incubated for 1 hour at 50° C., and then transformed into 50 μl of E. Coli JM109 using LB ampicillin medium to obtain pUC_RDN1L_URA3, pUC_RDN1L_LYS2 and PUCEco47RDNheadBamHI. PUCEco47RDNhyg1BamHI was prepared by cleaving pUCEco47RDNheadBamHI with the restriction enzymes Smal (Takara: 1085A) and NheI (Takara: 1241A) to obtain a plasmid backbone that was then ligated with hygromycin B resistant ribosomal RNA gene fragments (RDN-hyg1-1 fragment and RDN-hyg1-2 fragment). The RDN-hyg1-1 fragment and RDN-hyg1-2 fragment were prepared by treating the plasmid pRDN-hyg1 (Nucl Acids Res 2000, 28, 3524-) with the restriction enzymes PaeI (Thermo Fisher Scientific: FD0604) and NheI (Thermo Fisher Scientific: FD0973) or Smal (Thermo Fisher Scientific: FD0664) and purifying roughly 4 kb DNA fragments.

2.5 μl of a solution containing 10 ng of the plasmid backbone fragment obtained by Smal and NheI treatment, 25 ng of the RDN-hyg1-1 fragment and 25 ng of the RDN-hyg1-2 fragment was mixed with 2.5 μl of 2×Quick Ligation reaction buffer and 0.5 μl of Quick T4 DNA ligase and then left for 30 minutes at room temperature, after which 2 μl of the ligation reaction solution was transformed into 50 μl of E. Coli JM109 using LB ampicillin medium to obtain PUCEco47RDNhyg1BamHI.

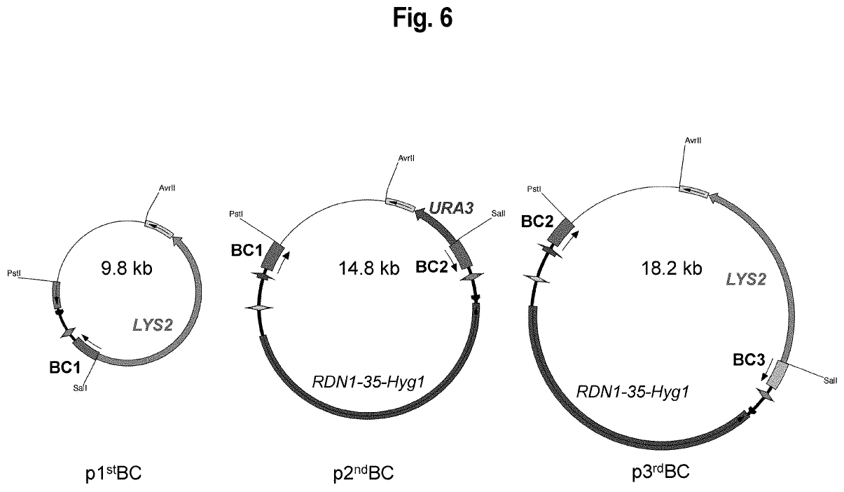

The vectors p1^{st}BC, p2^{nd}BC and p3^{rd}BC (FIG. 6) for use in incorporating the barcodes were prepared by mixing 2.5 μl of NEBuilder Master Mix with 2.5 μl of a mixture of 0.01 pmol each of the barcode sequence fragment, the ribosomal RNA gene fragment and a plasmid backbone obtained by treating pUC_RDN1L_LYS2 or pUC_RDN1L_URA3 with the restriction enzymes SalI (Thermo Fisher Scientific: FD0644) and PstI (Thermo Fisher Scientific: FD0614), performing an assembly reaction for 1 hour at 50° C., and transforming 50 μl of E. Coli JM109 in LB ampicillin plate medium at 30° C.

Using the respective barcode plasmids pBC1, pBC2 and pBC3 as templates, each barcode sequence was amplified by PCR for 2 minutes at 94° C. followed by 33 cycles of [10 seconds at 98° C., 20 seconds at 49° C., 1 minute at 68° C.] using the oligo-DNA primer sets OLI040 and OLI041 (BC_SalI fragment) and OLI044 and OLI045 (BC_PstI fragment), and then separated and purified by agarose gel electrophoresis. The ribosomal RNA gene-terminal RDN1-end fragment was amplified using the plasmid pUCEco47RDNheadBamHI as a template by performing PCR for 2 minutes at 94° C. followed by 33 cycles of [10 seconds at 98° C., 20 seconds at 49° C., 2 minutes at 68° C.] using the oligo-DNA primers OLI042 and OLI043, and then separated and purified by agarose gel electrophoresis.

For the full-length ribosomal RNA gene fragment RDNhyg1, the plasmid pUCEco47RDNhyg1BamHI was treated with the restriction enzymes AfeI (New England Biolabs: R0652) and BamHI (New England Biolabs: R3136S), and a roughly 9.1 kb DNA fragment was separated and purified by agarose gel electrophoresis. The vector p1^{st}BC was prepared by assembling the SalI-BC1 fragment, RDN1-end fragment and pUC_RDN1L_LYS2 backbone fragment (FIG. 6, left). The vector 2^{nd} BC was prepared by assembling the PstI-BC1 fragment, SalI-BC2 fragment, RDNhyg1 fragment and pUC_RDN1L_URA3 backbone fragment (FIG. 6, center). The vector p3^{rd}BC was prepared by assembling the PstI-BC2 fragment, SalI-BC3 fragment, RDNhyg1 fragment and pUC_RDN1L_LYS2 backbone fragment (FIG. 6, right).

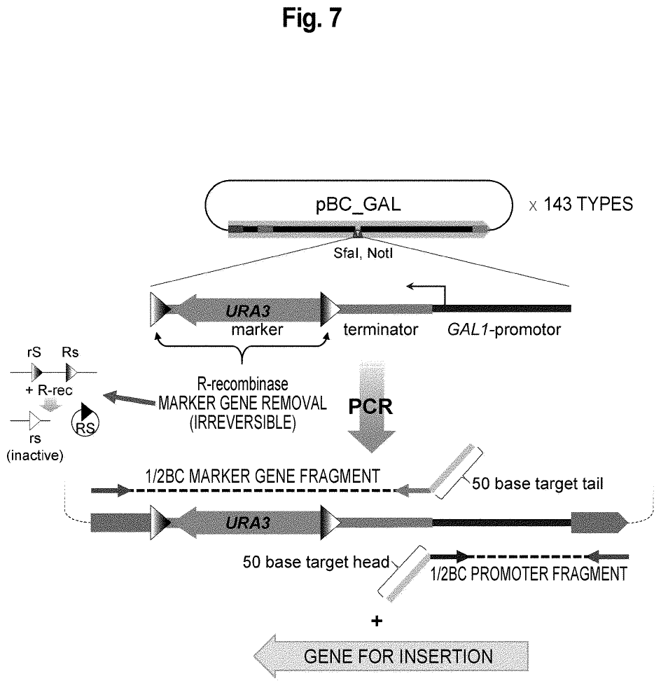

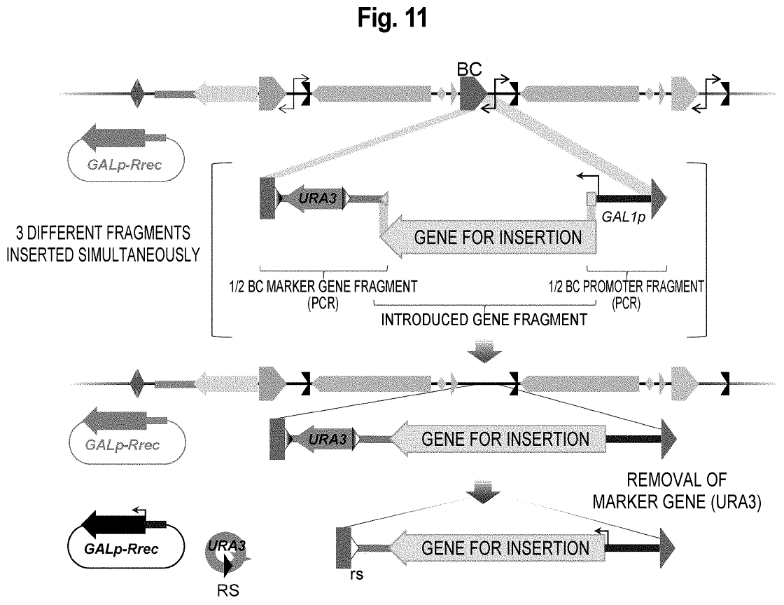

An R-recombinase expressing plasmid YEp181GALpR (FIG. 11: GALp-Rec), a galactose inducible expression cassette plasmid pUC_AsiSI-GAL1pCYC1t-rSURA3Rs-NotI, and expression cassette barcode plasmids pBC1GAL, pBC2GAL and pBC3GAL (FIG. 7, top) were prepared as vectors for systematic incorporation of the target gene.

YEp181GALpR was prepared by inserting a galactose-inducible R-recombinase gene fragment (GALpR fragment) into the restriction enzyme site SalI-EcoRI of the budding yeast shuttle vector YEplac181 (Gene 1988, 74, 527-). For the GALpR fragment, PRINT (Science 276, 1997, 806-) was treated with the restriction enzymes EcoRI (New England Biolabs: R3136S), SalI (New England Biolabs: R3138S), PstI (New England Biolabs: R3140S) and PfIFI (New England Biolabs: R0595S), and a roughly 2.6 kb DNA fragment was separated and purified by agarose gel electrophoresis.

pUC_AsiSI-GAL1pCYC1t-rSURA3Rs-NotI was prepared by assembling a GAL1p fragment, CYC1t fragment, rS-URA3-Rs fragment and pUC-vector fragment. Using a plasmid pAG413GAL-Ago2 (NAR 2001, 39, e43) derived from the pAG plasmid series (Yeast 2007, 24, 913-) as a template, the GAL1p fragment and CYC1t fragment were each amplified by PCR for 2 minutes at 94° C. followed by 31 cycles of [10 seconds at 98° C., 15 seconds at 49° C., 1 minute 5 seconds at 68° C.] using the respective oligo-DNA primer sets OLI059 and OLI060 (GAL1p fragment) and OLI061 and OLI062 (CYC1t fragment), and separated and purified by agarose gel electrophoresis.

The rS-URA3-Rs fragment was amplified by PCR for 2 minutes at 94° C. followed by 31 cycles of [10 seconds at 98° C., 15 seconds at 49° C., 1 minute 5 seconds at 68° C.] using the oligo-DNA primers OLI063 and OLI064 with YIplac211 as the template, and separated and purified by agarose gel electrophoresis. pBC1GAL, pBC2GAL and pBC3GAL were each prepared by inserting a galactose inducible expression cassette fragment (GAL1pCYC1t-rSURA3Rs fragment) into the restriction enzyme site Sfal-NotI of the respective barcode plasmids pBC1, pBC2 and pBC3.

For the GAL1pCYC1t-rSURA3Rs fragment, the plasmid pUC_AsiSI-GAL1pCYC1t-rSURA3Rs-NotI was treated with the restriction enzymes SfaAI (Thermo Fisher Scientific: FD2094) and NotI (Thermo Fisher Scientific: FD0593), and a roughly 2 kb DNA fragment was separated and purified by agarose gel electrophoresis.

To reconstruct an RNA interference mechanism using an in-yeast experimental system, a reporter gene vector YIp128PDA1pGFP expressing a GFP gene from Aequorea victoria and a hairpin RNA expression vector YIp211TEF1p-GFPhairpin comprising a GFP gene fragment were prepared. YIp128PDA1pGFP was prepared by assembling a GFP gene fragment (NLS-GFP-adh1t fragment), a budding yeast PDA1 gene promoter fragment (PDA1p fragment) and a plasmid backbone obtained by treating the budding yeast shuttle vector YIplac128 (Gene 1988, 74, 527) with EcoRI (Thermo Fisher Scientific: FD0274) and HindIII (Thermo Fisher Scientific: FD0504).

YIp211TEF1p-GFPhairpin was prepared by assembling a plasmid backbone obtained by treating the budding yeast shuttle vector YIplac211 with EcoRI (FD0274) and HindIII (FD0504), a budding yeast TEF1 gene promoter fragment (TEF1p fragment), a pfg1 fragment comprising part of a GFP gene, a gfp2 fragment, and a budding yeast CYC1 gene terminator fragment (cyclt fragment). Using genome DNA of the budding yeast strain BY4741 as a template, the PDA1p fragment, TEF1p fragment and cyc1t fragment were each amplified by PCR for 2 minutes at 94° C. followed by 30 cycles of [10 seconds at 98° C., 20 seconds at 48° C., 1 minute at 68° C.] using the respective oligo-DNA sets OLI073 and OLI074, OLI065 and OLI066 and OLI071 and OLI072, and separated and purified by agarose gel electrophoresis.

Using the GFP cassette plasmid pKT127 (Yeast 2004, 661-) as a template, the NLS-GFP-adh1t fragment, pfg1 fragment and gfp2 fragment were each amplified by PCR for 2 minutes at 94° C. followed by 30 cycles of [10 seconds at 98° C., 20 seconds at 48° C., 1 minute at 68° C.] using the respective oligo-DNA sets OLI075 and OLI076, OLI067 and OLI068 and OLI069 and OLI070, and separated and purified by agarose gel electrophoresis.

### 2. Preparing Artificial Chromosome Vector

The budding yeast strains YTT399 (MATa his3Δ1 ura3Δ0 leu2Δ0 met15Δ0 lys2Δ0 fob1Δ0 RDN1-15) and YTT401 (MATα his3Δ1 ura3Δ0 leu2Δ0 met15Δ0 lys2Δ0 fob1Δ0 RDN1-15) were used as hosts having low-copy rDNA. Each barcode incorporation vector plasmid was cleaved by treatment with the restriction enzymes PstI (New England Biolabs: R3140S) and AvrII (New England Biolabs: R0174S), and introduced by transformation into the host yeast in the order p1stBC, p2ndBC and p3^{rd}BC.

Transformation was performed by first suspending about 2.5×10⁸ exponential growth phase cells in 45 μl of transformation solution [0.1 M lithium acetate (Wako: 123-01542), 15% polyethylene glycol 3350 (Sigma: P4338-500G), 278 μg/ml heat denatured salmon sperm DNA (Sigma: D1626)] comprising the vector fragments, and incubating for 1 hour at 30° C. After incubation at 30° C., 5 μl of dimethyl sulfoxide (Wako: 043-07216) was added to the transformation solution to resuspend the cells, which were then heat shocked for 15 minutes at 42° C., coated on selection medium, and cultured for 3 to 4 days at 30° C. to select cells having the barcodes introduced into rDNA.

Introduction of the p1^{st}BC and p3^{rd}BC vector fragments was performed with HC-Lys selection medium [0.17% YNB (BD Yeast Nitrogen Base w/o Amino Acids and Ammonium Sulfate: 233520), 2% D-glucose (Wako: 049-31165), 2% agar (BD Bacto Agar: 214010), 20 mg/L L-arginine (Wako: 017-04612), 60 mg/L L-tyrosine (Wako: 202-03562), 80 mg/L L-isoleucine (Wako: 121-00862), 50 mg/L L-phenylalanine (Wako: 161-01302), 100 mg/L L-glutamic acid (Wako: 070-00502), 100 mg/L L-aspartic acid (Wako: 010-04842), 150 mg/L L-valine (Wako: 228-00082), 200 mg/L L-threonine (Wako: 204-01322), 400 mg/L L-serine (Wako: 199-00402), 40 mg/L L-tryptophan (Wako: 204-03382), 60 mg/L L-leucine (Wako: 124-00852), 20 mg/L L-histidine (Wako: 084-00682), 20 mg/L L-methionine (Wako: 133-01602), 40 mg/L adenine sulfate (Nacalai Tesque: 01990-94), 20 mg/L uracil (Wako: 212-00062)].

Introduction of the p2^{nd}BC vector fragment was performed with HC-URA selection medium [0.17% YNB (BD Yeast Nitrogen Base w/o Amino Acids and Ammonium Sulfate: 233520), 2% D-glucose (Wako: 049-31165), 2% agar (BD Bacto Agar: 214010), 20 mg/L L-arginine (Wako: 017-04612), 60 mg/L L-tyrosine (Wako: 202-03562), 80 mg/L L-isoleucine (Wako: 121-00862), 50 mg/L L-phenylalanine (Wako: 161-01302), 100 mg/L L-glutamic acid (Wako: 070-00502), 100 mg/L L-aspartic acid (Wako: 010-04842), 150 mg/L L-valine (Wako: 228-00082), 200 mg/L L-threonine (Wako: 204-01322), 400 mg/L L-serine (Wako: 199-00402), 40 mg/L L-tryptophan (Wako: 204-03382), 60 mg/L L-leucine (Wako: 124-00852), 20 mg/L L-histidine (Wako: 084-00682), 20 mg/L L-methionine (Wako: 133-01602), 40 mg/L adenine sulfate (Nacalai Tesque: 01990-94), 120 mg/L L-lysine monohydrochloride (Nacalai Tesque: 20809-52)].

The artificial chromosome vector strains having the three introduced barcodes BC1, BC2 and BC3 were called YTT430 (YTT399+BC1,2,3-LYS2) and YTT431 (YTT401+BC1,2,3-LYS2).

### 3. Preparing Artificial Gene Amplification System

When an URA3-lacO cassette was inserted into the IGS1 region of a budding yeast previously isolated by Kobayashi et al and having only two copies of rDNA (Kobayashi et al., 2001), and rDNA amplification was induced by causing expression of the Fob1 protein, which binds to the replication fork barrier and activates recombination, the URA3-lacO cassette was also amplified to about 100 copies together with the rDNA.

Methods: (see Tables 5 to 7 for yeast strains, plasmids and primers)

The plasmid pAFS52 having 250 copies of the lacO repeat was treated with the restriction enzyme EcoRI, and re-ligated to reduce the number of lacO repeats to 50 copies and prepare pAFS52-lacO50. Using the primers TM3 and TM4, part of the IGS1 DNA of the rDNA was increased and then inserted into the restriction enzyme KpnI-XhoI site of pAFS52-lacO50 to prepare a plasmid pTM1. Part of the IGS2 of the rDNA was increased with the primers TM-Hind and TM-Sph, and inserted into the HindIII-SphI of pTM1 to prepare pTM2. The URA3 gene was amplified with primers TM7 and TM8 from the plasmid pJJ242, and inserted into the SphI-SalI of pTM2 to construct pTM-lacO50 having a URA3-lacO cassette.

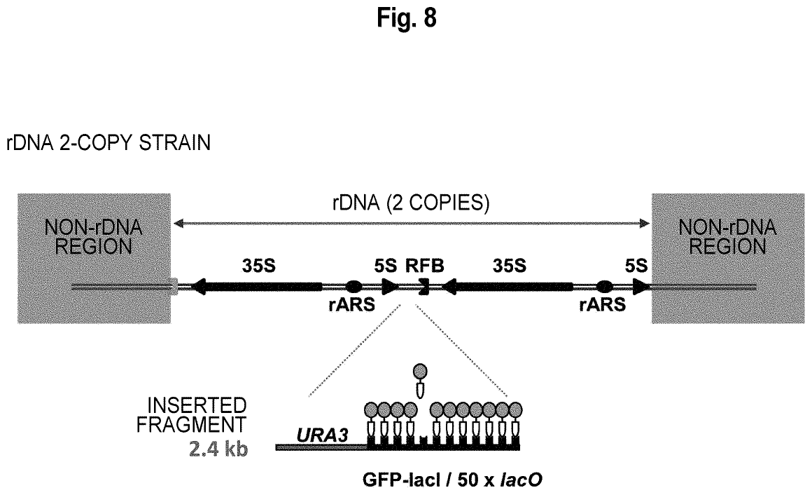

pTM-lacO50 was cleaved with Kpnl-HindIII and introduced into a yeast strain TAK201 having two copies of rDNA, and selection was performed with uracil-free synthetic selection medium (SG-Ura) to prepare a yeast strain having a URA3-lacO cassette in the IGS1 of the rDNA (FIG. 8). A pAFS144-ADE2 plasmid expressing lacI-GFP was further inserted into ade2, and selection was performed in adenine-free synthetic selection medium (SG-Ade) to perfect a yeast strain TMY1 in which amplification of living rDNA can be detected by fluorescence.

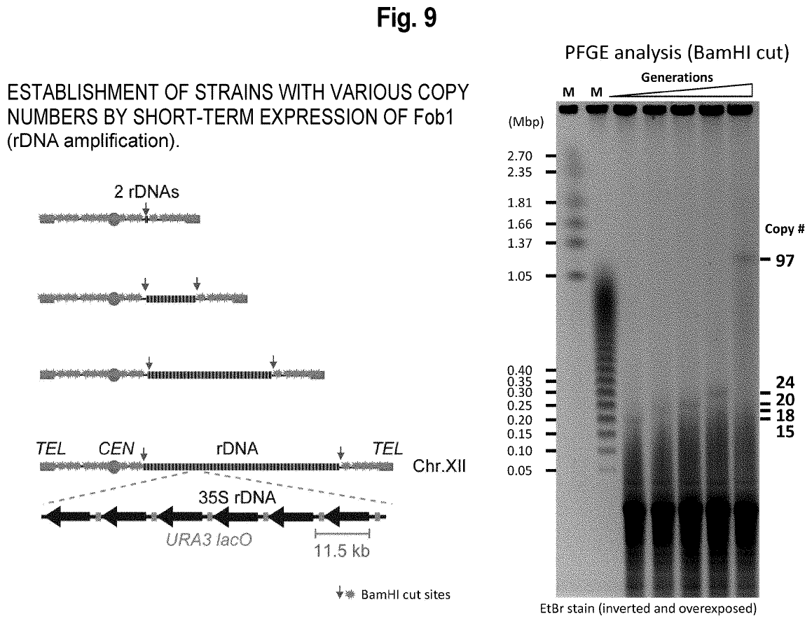

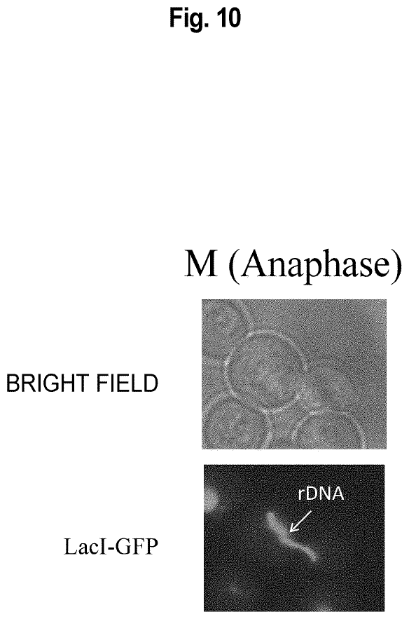

A plasmid pTAK101 expressing Fob1 was introduced into TMY1, which was then cultured in synthetic selection medium (SG-Ade, -Leu), and cells were harvested over time and mated to delete the Fob1 plasmid and stop rDNA amplification. The DNA was purified with the cells enveloped in agarose gel plugs, treated with the restriction enzyme BamHI having no recognition sequence in rDNA, and analyzed by pulse field electrophoresis (FIG. 9). Most yeast DNA is broken down into small fragments by BamHI, but because rDNA repeats are not cleaved by BamHI, they can be detected as large DNA. As a result, it was confirmed that the number of rDNA copies was amplified depending on the Fob1 expression time. Amplification of the URA3-lacO cassette together with the rDNA was also confirmed by fluorescent microscopy because lacO-LacIGFP binds to whole rDNA and emits fluorescence (FIG. 10).

TABLE 5

| Strain | Genotype | Reference |
|---|---|---|
| TAK201 | MAT a 1eu2-3, 112 trp1-1 | Mol Cell |
| | can1-100 ura3-1 ade2-1 | Biol., |
| | his3-11, 15 fob1D::HIS3 | (2001)21: |
| | (2 rDNA copies) pN0Y353 | 136-147. |
| TMY1 | Same as TAK201 except | Genes Cells, |
| | ade2::pAFS144-wtGFP (ADE2) | (2010) 16: |
| | rDNA::pTM-1ac050 (URA3) | 491-502 |

TABLE 6

| Plasmid | Contents | Reference |
|---|---|---|
| pAFS52 | Plasmid containing the | Curr Biol. |
| | 256x 1ac0 array | (1996) 6: |
| | | 1599-1608 |
| pAFS52- | Plasmid containing the | Genes Cells, |
| 1ac050 | 50x 1ac0 array | (2010) 16: |
| | | 491-502 |
| pTM1 | IGS1 fragment was inserted | Miyazaki et |
| | into the KpnI-XhoI site | al., (Genes |
| | of pAFS52-1ac050 | Cells, |
| | | (2010) 16: |
| | | 491-502 |
| pTM2 | IGS2 fragment was inserted | Genes Cells, |
| | into the HindIII-SphI | (2010) 16: |
| | site of pTM1 | 491-502 |
| pTM-1ac050 | Plasmid used for labeling | Genes Cells, |
| | the rDNA unit with 1ac0 | (2010) 16: |
| | | 491-502 |
| pAFS144 | Plasmid containing GFP- | Curr Biol. |
| | 1acI fragment | (1996) 6: |
| | | 1599-1608 |
| pAFS144-ADE2 | Plasmid containing wtGFP- | Genes Cells, |
| | 1acI and ADE2 fragment | (2010) 16: |
| | | 491-502 |
| pTAK101 | Yeplace181-F0B1 | Mol Cell |
| | | Biol., |
| | | (2001)21: |
| | | 136-147. |

TABLE 7

| Primer | Sequence | Reference |
|---|---|---|
| TM3 | 5′-ATCTCGAGTCCCATAACTAACCTACCAT-3′ | Genes Cells, |
| | (SEQ ID NO: 86) | (2010) |
| | | 16: 491-602 |
| TM4 | 5′-ACGGTACCAGTAAATGOCAGTTTCTAGG-3′ | Genes Cells, |
| | (SEQ ID NO: 87) | (2010) |
| | | 16: 491-602 |
| TM-Hind | 5′-CCCCAAGCTTACCTACCGACCAACTTTCAT-3′ | Genes Cells, |
| | (SEQ ID NO: 88) | (2010) |
| | | 16: 491-602 |
| TM-Sph | 5′-CACATGCATGCTTTAGCATAGGAAGCCAAGA-3′ | Genes Cells, |
| | (SEQ ID NO: 89) | (2010) |
| | | 16: 491-502 |
| TM7 | 5′-CACATGCATGCAGCTTTTCAATTCATCT-3′ | Genes Cells, |
| | (SEQ ID NO: 90) | (2010) |
| | | 16: 491-602 |
| TM8 | 5′-ACGCGTCGACATCATTACGACCGAGATTCC-3′ | Genes Cells, |
| | (SEQ ID NO: 91) | (2010) |
| | | 16: 491-602 |

### 4. Establishing In-Yeast Experimental System

A gene silencing mechanism by RNAi via short double-stranded RNA (siRNA) does not exist in the budding yeast S. cerevisiae, which is the donor of the artificial chromosome vector. As for the RNAi mechanisms seen in human cells, a sequence-specific target RNA cleavage reaction can be reconstructed by mixing purified Dicer (HsDicer), Argonaute (HsAgo2) and TRBP (HsTRBP) with double-stranded RNA in a test tube (Nature 2006, 436, 740-). It has also been reported that silencing of target genes was observed when antisense RNA having introduced HsDicer, HsAgo2 and HsTRBP was transcribed into S. cerevisiae cells (NAR 2011, 39, e43), but actual production of siRNA has not been confirmed.

We therefore introduced HsDicer, HsAgo2 and HsTRBP into a chromosome vector, and investigated whether silencing of a target gene by hairpin RNA-dependent human RNAi occurs in an in-yeast experimental system. HsTRBP, HsAgo2 and HsDicer were each introduced together with an URA3 marker gene downstream from a galactose inducible promoter in BC1, BC2 and BC3, respectively, and after gene introduction the marker gene was removed with R-recombinase.

For each gene introduction, a human RNAi gene fragment excised from a plasmid, and a 1/2BC promoter fragment and 1/2BC marker gene fragment which had been amplified by PCR of 2 minutes at 94° C. followed by 33 cycles of [10 seconds at 98° C., 15 seconds at 49° C., 1 minute at 68° C.] using pBC1^{˜}3GAL as the template, were separated and purified by agarose gel electrophoresis and transformed into a yeast strain (YTT431-R) obtained by introducing the R-recombinase expression plasmid YEp181GALpR into the chromosome vector strain YTT431 (FIG. 11).

For the HsTRBP gene, a 1/2BC promoter fragment and a 1/2BC marker gene fragment were synthesized with the oligo-DNA primer sets OLI013 and OLI086 and OLI014 and OLI087, respectively, using pBC1GAL as the template, and introduced into the barcode BC1 using an HsTRBP fragment obtained by treating the HsTRBP donor plasmid pAG415Gal-TRBp (NAR 2011, 39, e43) with the restriction enzymes BamHI (Thermo Fisher Scientific: FD0544) and Eco321 (Thermo Fisher Scientific: FD0303). For the HsAgo2 gene, a 1/2BC promoter fragment and a 1/2BC marker gene fragment were synthesized with the oligo-DNA primer sets OLI013 and OLI084 and OLI014 and OLI085, respectively, using pBC2GAL as the template, and introduced into the barcode BC2 using an HsAgo2 fragment obtained by treating the HsAgo2 donor plasmid pAG413Gal-Ago2 (NAR 2011, 39, e43) with the restriction enzymes BamHI (Thermo Fisher Scientific: FD0544) and EcoRI (Thermo Fisher Scientific: FD0274).

For the HsDicer gene, a 1/2BC promoter fragment and a 1/2BC marker gene fragment were synthesized with the oligo-DNA primer sets OLI013 and OLI082 and OLI014 and OLI083, respectively, using pBC3GAL as the template, and introduced into the barcode BC3 using an HsDicer fragment obtained by treating the HsDicer donor plasmid pAG416Gal-Dicer (NAR 2011, 39, e43) with the restriction enzymes HindIII (Thermo Fisher Scientific: FD0504) and BcuI (Thermo Fisher Scientific: FD1253). Each gene was selected with HC-Ura Leu plate medium [0.17% YNB (BD Yeast Nitrogen Base w/o Amino Acids and Ammonium Sulfate: 233520), 2% D-glucose (Wako: 049-31165), 2% agar (BD Bacto Agar: 214010), 20 mg/L L-arginine (Wako: 017-04612), 60 mg/L L-tyrosine (Wako: 202-03562), 80 mg/L L-isoleucine (Wako: 121-00862), 50 mg/L L-phenylalanine (Wako: 161-01302), 100 mg/L L-glutamic acid (Wako: 070-00502), 100 mg/L L-aspartic acid (Wako: 010-04842), 150 mg/L L-valine (Wako: 228-00082), 200 mg/L L-threonine (Wako: 204-01322), 400 mg/L L-serine (Wako: 199-00402), 40 mg/L L-tryptophan (Wako: 204-03382), 20 mg/L L-histidine (Wako: 084-00682), 20 mg/L L-methionine (Wako: 133-01602), 40 mg/L adenine sulfate (Nacalai Tesque: 01990-94), 120 mg/L L-lysine monohydrochloride (Nacalai Tesque: 20809-52)]. Cells in which gene introduction was confirmed were then cultured overnight at 30° C. in 1 ml of HCGal-Leu medium [0.17% YNB (BD Yeast Nitrogen Base w/o Amino Acids and Ammonium Sulfate: 233520), 2% D-galactose (Wako: 075-00035), 20 mg/L L-arginine (Wako: 017-04612), 60 mg/L L-tyrosine (Wako: 202-03562), 80 mg/L L-isoleucine (Wako: 121-00862), 50 mg/L L-phenylalanine (Wako: 161-01302), 100 mg/L L-glutamic acid (Wako: 070-00502), 100 mg/L L-aspartic acid (Wako: 010-04842), 150 mg/L L-valine (Wako: 228-00082), 200 mg/L L-threonine (Wako: 204-01322), 400 mg/L L-serine (Wako: 199-00402), 40 mg/L L-tryptophan (Wako: 204-03382), 20 mg/L L-histidine (Wako: 084-00682), 20 mg/L L-methionine (Wako: 133-01602), 40 mg/L adenine sulfate (Nacalai Tesque: 01990-94), 120 mg/L L-lysine monohydrochloride (Nacalai Tesque: 20809-52), 20 mg/L uracil (Wako: 212-00062)], and the URA3 gene was removed with R-recombinase (FIG. 11, removal of URA3).

The URA3-depleted cells were selected at 30° C. in HC-Leu+5FOA plate medium [0.17% YNB (BD Yeast Nitrogen Base w/o Amino Acids and Ammonium Sulfate: 233520), 2% D-glucose (Wako: 049-31165), 2% agar (BD Bacto Agar: 214010), 20 mg/L L-arginine (Wako: 017-04612), 60 mg/L L-tyrosine (Wako: 202-03562), 80 mg/L L-isoleucine (Wako: 121-00862), 50 mg/L L-phenylalanine (Wako: 161-01302), 100 mg/L L-glutamic acid (Wako: 070-00502), 100 mg/L L-aspartic acid (Wako: 010-04842), 150 mg/L L-valine (Wako: 228-00082), 200 mg/L L-threonine (Wako: 204-01322), 400 mg/L L-serine (Wako: 199-00402), 40 mg/L L-tryptophan (Wako: 204-03382), 20 mg/L L-histidine (Wako: 084-00682), 20 mg/L L-methionine (Wako: 133-01602), 40 mg/L adenine sulfate (Nacalai Tesque: 01990-94), 120 mg/L L-lysine monohydrochloride (Nacalai Tesque: 20809-52), 20 mg/L uracil (Wako: 212-00062), 1 g/L 5-fluoroorotic acid (fluoroChem: 003234)].

The resulting transfected cells were used for introducing other human RNAi genes, to prepare single-expressing strains for HsDicer (YTT431-D), HsAgo2 (YTT431-A) and HsTRBP (YTT431-T) as well as two-gene expressing strains (YTT431-DA, -DT, -AT) and a three-gene expressing strain (YTT431-DAT).

To provide a GFP reporter gene for evaluating gene suppression by the human RNAi mechanism, YIp128PDA1pGFP was treated with the restriction enzyme AccI (New England Biolabs: R0161S), and inserted into the terminator region of the ADH1 gene of the chromosome vector strain YTT430 (GFP reporter strain: YTT430-GFP). To provide a hairpin RNA gene necessary for inducing the human RNAi mechanism, the GFP hairpin vector YIp211TEF1p-GFPhairpin was treated with the restriction enzyme HindIII (Thermo Fisher Scientific: FD0504), and inserted into the terminator region of the CYC1 gene of YTT430-GFP (GFP hairpin strain: YTT430-GFPhp).

The alpha mating-type strains YTT431 and YTT431-D˜DAT were mated with the a-mating type YTT430-GFP and YTT430-GFPhp, to prepare diploid strain sets expressing the human RNAi gene, the GFP reporter gene and GFP hairpin RNA.

To express the human RNAi gene, the cell extract, in which a diploid strain obtained by mating YTT431, YTT431-D^{˜}DAT and YTT430-GFPhp was cultured to the exponential growth phase at 30° C. in HCGal-Leu medium [0.17% YNB (BD Yeast Nitrogen Base w/o Amino Acids and Ammonium Sulfate: 233520), 2% D-galactose (Wako: 075-00035), 20 mg/L L-arginine (Wako: 017-04612), 60 mg/L L-tyrosine (Wako: 202-03562), 80 mg/L L-isoleucine (Wako: 121-00862), 50 mg/L L-phenylalanine (Wako: 161-01302), 100 mg/L L-glutamic acid (Wako: 070-00502), 100 mg/L L-aspartic acid (Wako: 010-04842), 150 mg/L L-valine (Wako: 228-00082), 200 mg/L L-threonine (Wako: 204-01322), 400 mg/L L-serine (Wako: 199-00402), 40 mg/L L-tryptophan (Wako: 204-03382), 20 mg/L L-histidine (Wako: 084-00682), 20 mg/L L-methionine (Wako: 133-01602), 40 mg/L adenine sulfate (Nacalai Tesque: 01990-94), 120 mg/L L-lysine monohydrochloride (Nacalai Tesque: 20809-52), 20 mg/L uracil (Wako: 212-00062)], was separated by SDS-PAGE and verified by Western blotting.

Yeast cells that had been collected to OD₆₀₀=1.0 were suspended in 250 μl of sterile water, 37.5 μl of alkali denaturing solution (1 N sodium hydroxide (Wako: 198-13765), 7.5% 2-mercaptoethanol (Wako: 137-07521)] was added, and the cells were cooled for 5 minutes on ice, after which 37.5 μl of 50% trichloroacetic acid solution (Wako: 208-08081) was added and the cells were further cooled for 5 minutes on ice to precipitate total proteins. The proteins were pelleted by centrifugation for 5 minutes at 4° C., 10,000 g, suspended in 30 μl of 1×sample buffer [75 mM Tris (Sigma-Aldrich: T1503), 100 mM 2-mercaptoethanol (Wako: 137-07521), 2% sodium dodecylsulfate (Wako: 196-08675), 5% glycerin (Wako: 075-00611) and 0.001% BPB (Wako: 029-02912)], and denatured for 5 minutes at 65° C. to obtain a sample solution for Western blotting.

5 μl of the sample solution was subjected to SDS-PAGE using 5 to 20% polyacrylamide gel (ATTO: EHR-T/R520L) with a slab-type electrophoresis unit (ATTO: WSE-1150 PageRunAce). Electrophoresis was performed using 1×running buffer [25 mM Tris (Sigma-Aldrich: T1503), 192 mM glycine (Wako: 077-00735), 0.1% sodium dodecyl sulfate (Wako: 196-08675)] at a constant current of 10 mA for 30 minutes and 20 mA for 90 minutes. The proteins separated by SDS-PAGE were transferred with a mini transblot cell (BIO-RAD: 1703930JA) to a PVDF membrane (Immobilon-P Merck-Millipore: PVH00010) using transfer buffer [0.302% Tris (Sigma-Aldrich: T1503), 1.44% glycine (Wako: 077-00735), 10% methanol (Wako: 137-01823)] at 4° C. at a constant current of 150 V for 30 minutes.

The receiving membrane was immersed in PBS-T [140 mM sodium chloride, 2.7 mM potassium chloride, 10 mM phosphoric acid, 0.05% Tween 20 (Sigma-Aldrich: P7949)] containing 5% skim milk (Wako: 190-12865), and shaken for 1 hour at room temperature to perform blocking. The antibodies for detecting tubulin, HsAgo2 and HsTRBP were labeled with a Peroxidase Labeling Kit-NH2 (Dojindo: LK11). For the HsDicer, HsAgo2, HsTRBP and tubulin, the anti-Dicer antibody (Santa Cruz Biotechnology: sc-30226) was diluted 1/300, the anti-Ago2 antibody (clone 11A9 Merck-Millipore: MABE253) was diluted 1/1,000, the anti-TRBP antibody (clone 46D1 Funakoshi: bsm-50266M) was diluted 1/10,000, and the anti-Tubulin Alpha antibody (clone YL1/2 AbD Serotec: MCA77G) was diluted 1/2,500 with PBS-T containing 5% skim milk, and shaken overnight at 4° C. to perform detection (primary antibody binding). The primary antibody-bound membrane was washed twice by shaking for 30 minutes at room temperature with PBS-T. For the HsDicer detection, the washed membrane was further shaken for 1 hour at room temperature with a peroxidase-labeled anti-rabbit IgG antibody (GE Health Care: NA934) diluted 1/10,000 with PBS-T containing 0.5% skim milk, and then washed twice by shaking for 30 minutes at room temperature in PBS-T.

The proteins were detected with a chemiluminescence imaging system (Vilber-Lourmat: Fusion SL) after the washed membrane had been first incubated for 5 minutes at room temperature with a chemiluminescent substrate (Merck-Millipore: WBKLS0500). The results of Western blotting confirm that each human RNAi protein is expressed in an amount that can be adequately detected in cells from the genes of the introduced barcode sequences.

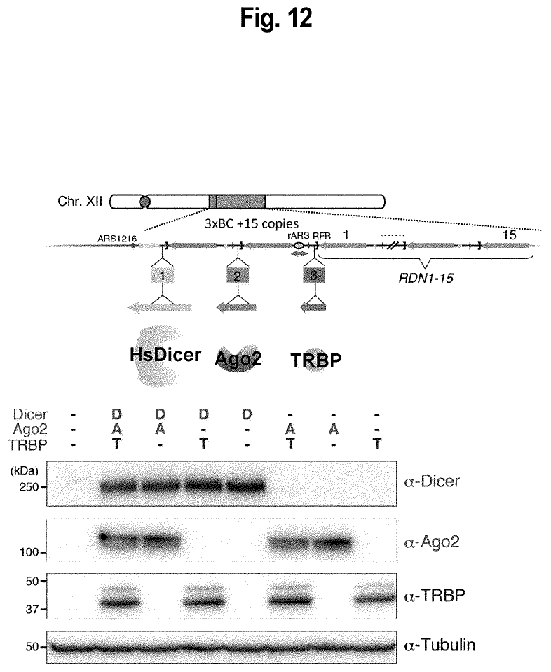

To determine whether the human RNAi mechanism functions in an in-yeast experimental system, total RNA was extracted from a diploid strain expressing a human RNAi gene, a GFP reporter gene and GFP hairpin RNA, and hairpin RNA-derived siRNA production and reporter gene transcripts were analyzed by the Northern method. The amount of reporter gene transcripts was determined by reverse transcription quantitative PCR (RT-qPCR), and gene silencing by the human RNAi mechanism was analyzed (FIG. 12).

For the total RNA, roughly 5×10⁸ cells that had been cultured to the exponential phase in YPGal [2% tryptone (BD Bacto peptone: 211677), 1% yeast extract (BD Bacto Yeast Extract: 212750), 2% D-galactose (Wako: 075-00035)] were washed in cool sterile water, suspended in 400 μl of TES solution [10 mM Tris-CI pH 7.5 (Tris, Sigma-Aldrich: T1503/hydrochloric acid, Wako: 080-01066), 10 mM EDTA-Na pH 8.0 (EDTA2Na, Dojindo: 345-01865/NaOH, Wako: 198-13765), 0.5% sodium dodecyl sulfate (Wako: 196-08675)] and 400 μl of acidic phenol pH 4.0 (Phenol, Wako: 160/12725/glycine, Wako: 077-00735/hydrochloric acid, Wako: 080-01066), and extracted by heating and shaking for 30 minutes at 65° C. with a heat mixer (Eppendorf ThermoMixer, C Eppendorf: 5382000023/Eppendorf SmartBlock, Eppendorf: 5362000035). The extracted 400 μl RNA solution was centrifuged for 10 minutes at 4° C., 20,000 g, the water layer fraction was collected and purified by acidic phenol/chloroform (1:1, pH 4.0) [acidic phenol pH 4.0/chloroform (Wako: 308-026069)] extraction, and 50 μl of 3 M sodium acetate (pH 5.2), 1 μl of a coprecipitant for alcohol precipitation (Ethachinmate, Nippon Gene: 312-01791) and 1 ml of ethanol (Wako: 057-00456) were added, and the mixture was stored overnight at −20° C. to perform ethanol precipitation.

The total RNA was centrifuged for 40 minutes at 4° C., 20,000 g, washed and dried with 80% ethanol (Wako: 057-00456), and dissolved in sterile water to obtain a sample for Northern analysis. In Northern analysis for detecting siRNA, 5 μg of the total RNA (3 μl solution) was mixed with 3 μl of formamide (Wako: 066-02301), heat denatured for 10 minutes at 65° C., and separated by electrophoresis with denatured acrylamide gel [0.5×TBE, 8 M urea (Wako: 219-00175), 12% acrylamide (BIO-RAD: 1610144), 0.1% ammonium persulfate (Wako: 018-03282), 0.1% tetramethylene diamine (Wako: 202-04003).

Electrophoresis was performed at room temperature for 2 hours with 0.5×TBE at a constant current of 7 mA using a slab-type electrophoresis machine (Bio-craft: BE-140G). The separated RNA was transferred (siRNA blot) to a nylon membrane (Hybond-N+, GE Health Care: RPN303B) by electrophoresis for 2 hours at 4° C., constant current 400 mA using 0.5×TBE and a semidry transfer machine (Trans-Blot SD Cell, Bio-Rad: 1703940JA) with extra thick filter paper (Bio-Rad: 1703968). The siRNA blot was crosslinked at 120,000 μJ/cm² with a UV crosslinker (Stratagene: StrataLinker 1800), washed with 5×SSC [75 mM sodium citrate (Wako: 191-01785), 0.75 M sodium chloride (Wako: 191-01665)], dried, and stored.

For Northern analysis of the siRNA, the siRNA blot was wetted with sterile water, pre-hybridized for 2 hours at 42° C. in 10 ml of Rapid-Hyb buffer (GE Health Care: RPN1635), hybridized overnight at 25° C. after addition of 5 pmol of an oligo-DNA probe terminally labeled with ³²P, and washed twice for 10 minutes at room temperature with a washing buffer 1 (5×SSC, 0.1% sodium dodecyl sulfate), once for 10 minutes at room temperature with a washing buffer 2 (2×SSC, 0.1% sodium dodecyl sulfate) and once for 10 minutes at 42° C. with the washing buffer 2 (2×SSC, 0.1% sodium dodecyl sulfate), after which the siRNA blot was exposed on an imaging plate and the siRNA signal was detected with an FLA7000 (GE Health Care). To label the oligo-DNA probe, 5 pmol (16.5 μl) of DNA oligonucleotide prepared from a mixture with an equal amount of siGFP01^{˜}13 was mixed with 3 μl of T4 polynucleotide kinase (Takara: 2021S), 3 μl of 10×PNK buffer (Takara) and 7.5 μl of gamma-³²P-ATP (6000Ci/mmol, Perkin Elmer: NEG502Z) and then incubated for 30 minutes at 37° C., 7.5 μl of 50 mM EDTA (pH 8.0) was added, and the mixture was heat denatured (95° C., 5 minutes) and purified in a microspin column G-25 (GE Health Care: 27532501).

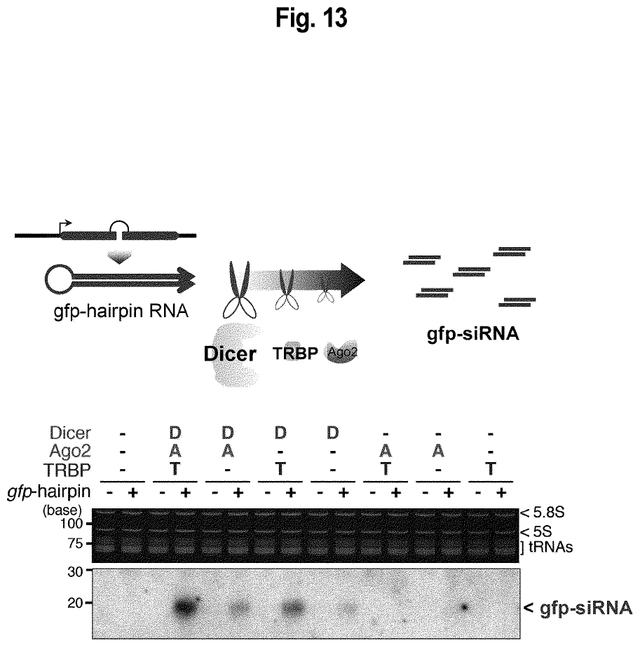

In Northern analysis of the siRNA, siRNA corresponding to the GFP sequence was detected in samples of cells expressing HsDicer and GFP hairpin RNA, and the amount of siRNA was shown to be greater in cells co-expressing HsTRBP and HsAgo2 in addition to HsDicer (FIG. 13). This suggests that the function of HsTRBP and HsAgo2 of assisting the working of HsDicer in vitro (Nature 2005, 436, 740-) can be reproduced in an in-yeast experiment.

Detection of the GFP reporter gene transcription product and hairpin RNA was performed by ordinary Northern analysis. The DNA probes were labeled with 100 ng of gfp2 fragments, 5 μl of alpha-³²P-dCTP (3000Ci/mmol, Perkin Elmer: NEG513H) and a random primer labeling kit (Takara: 6045), and purified with a G-50 Microspin Column (GE Health CAre: 28903408).

For the Northern blot, 5 μg of total RNA (3 μl solution) was mixed with 4 μl of formamide, 2 μl of formaldehyde solution (Wako: 061-00416), 1 μl of 10×MOPS buffer [0.2 M MOPS-NaOH pH 7 (MOPS, Dojindo: 345/01804/sodium hydroxide, Wako: 198-13765), 2 mM sodium acetate, 10 mM EDTA] and 1 μl of 400 μg/ml ethidium bromide (Wako: 315-90051), and denatured for 10 minutes at 65° C., and the product was separated by electrophoresis at constant voltage 100 V for 1 hour and constant voltage 150 V for 45 minutes in denatured agarose gel (1.2% Star Agarose, 1×MOPS, 6% formaldehyde) and 1×MOPS denaturing buffer (1×MOPS, 6% formaldehyde) using a Submarine electrophoresis system (Biocraft: BE-527).

The gel of the separated RNA was washed for 30 minutes in sterile water, and transferred by overnight capillary transfer using 10×SSC to a nylon membrane (Hybond-N+, GE Health Care: RPN303B) (RNA blot). The RNA blot was crosslinked at 120,000 μJ/cm² with a UV crosslinker (Stratagene: StrataLinker 2400), washed with 5×SSC, and dried and stored.

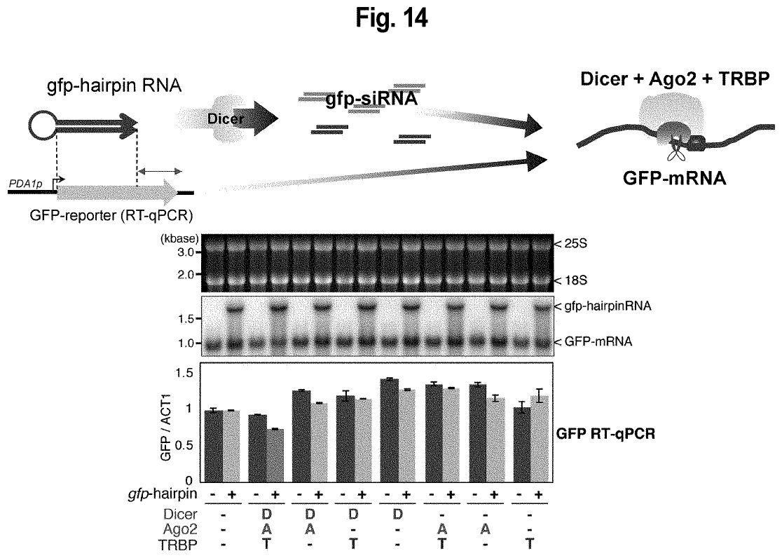

For the Northern analysis, the RNA blot was pre-hybridized for 2 hours at 65° C. in 10 ml of Rapid-Hyb buffer (GE Health Care: RPN1635), hybridized for 2 hours at 65° C. after addition of a gfp2 fragment probe labeled with ³²P, and washed once for 30 minutes at 65° C. with the washing buffer 2 (2×SSC, 0.1% sodium dodecyl sulfate) and twice for 30 minutes at 65° C. with a washing buffer 3 (0.1×SSC, 0.1% sodium dodecyl sulfate), after which the RNA blot was exposed on an imaging plate and the signals of the GFP reporter gene RNA and GFP hairpin RNA were detected with an FLA7000 (GE Health Care). The amount of GFP hairpin RNA in HsDicer-expressing cells in which siRNA derived from GFP hairpin RNA was detected was equivalent to that found in cells expressing no HsDicer, suggesting that degradation of GFP hairpins by HsDicer is less efficient than synthesis of GFP hairpin RNA (FIG. 14).

The RNA of the GFP reporter gene was also slightly diminished in cells expressing all four of GFP hairpin RNA, HsDicer, HsAgo2 and HsTRBP, suggesting that the human RNAi mechanism functions through hairpin RNA expression, allowing for the possibility of gene silencing.

The GFP reporter gene RNA was analyzed quantitatively using RT-qPCR. RT-qPCR was performed using a quantitative PCR system (Illumina: Eco Real Time PCR System) with a RT-qPCR kit (KAPA SYBR FAST One-Step qRT-PCR Kit, KAPA Biosystem: KK4650). The reaction solution for RT-qPCR was 12.5 μl of a mixture of qPCR Master Mix (1×), 0.2 μM of each oligo-DNA primer, KAPA RTMix (1×), ROX low Dye (1×) and 0.8 ng/μl of total RNA. As an internal standard, ACT1 gene RNA was analyzed by RT-qPCR for 10 minutes at 42° C. and 3 minutes at 95° C. followed by 40 cycles of [5 seconds at 95° C., 20 seconds at 50° C., 30 seconds at 72° C.] using 20 ng of total RNA and the oligo-DNA primer set OLI1126 and OLI1127.

The GFP reporter gene RNA was analyzed by performing RT-qPCR for 10 minutes at 42° C. and 3 minutes at 95° C. followed by 40 cycles of [5 seconds at 95° C., 20 seconds at 52° C., 30 seconds at 72° C.], using 20 ng of total RNA and the oligo-DNA primer set pPCRsplitGFPFw and pPCRsplitGFPRv. Each reaction was corrected with ROX low Dye, after which the cycle value (Ct value) was determined, and expression of the GFP reporter gene was analyzed quantitatively by the comparative Ct method using the Ct value of the ACT1 gene RNA and the Ct value of the GFP reporter gene RNA. As in the Northern analysis, in the RT-qPCR analysis the RNA of the GFP reporter gene was slightly diminished in cells expressing all four of GFP hairpin RNA, HsDicer, HsAgo2 and HsTRBP, confirming that the human RNAi mechanism functions in an in-yeast experiment.

### 5. Stabilization of Artificial Chromosome

When an artificial chromosome vector with an even greater number of barcodes was prepared by the methods of 2. above, the ribosomal RNA gene became unstable when the number of BACs exceeded 10, and there was a clear risk of copy loss. When the SIR2 gene was introduced upstream from the TRP1 gene on chromosome 4 of the host, not only did barcode insertion efficiency increase, but the ribosomal RNA gene was stabilized, and copy loss decreased. The protocols for introducing the SIR2 gene are shown below.

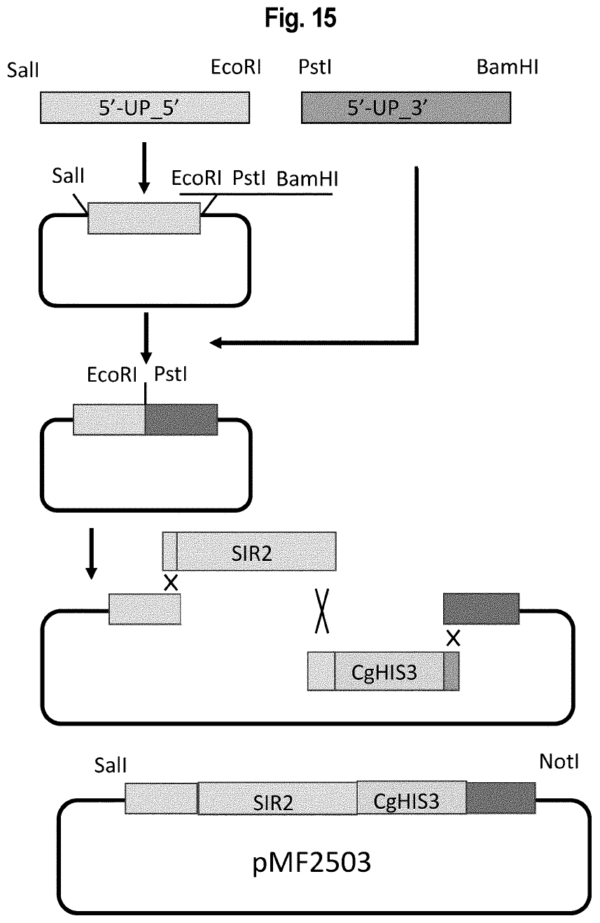

Using chromosomal DNA of the budding yeast BY4741 strain as a template, regions between the centromere and the TRP1 gene on the budding yeast chromosome 4 are amplified by PCR using the primers MN2509 (Table 1) and MN2510 and the primers MN2511 and MN2512, respectively. As shown in FIG. 15, the respective regions are called 5′-UP_5′ (about 250 base pairs) and 5′-UP_3′ (about 410 base pairs).

The 5′-UP_5′ fragment was cleaved with the restriction enzymes SalI and EcoRI, and inserted into the plasmid pTA2, which had already been cleaved with the same enzymes. The resulting recombinant plasmid was cleaved with the restriction enzymes PstI and BamHI. Naturally the 5′-UP_3′ fragment was then cleaved with PstI and BamHI and inserted into the same plasmid.

The resulting recombinant plasmid pTA-5′-UP_5′+3′ (FIG. 15) was cleaved with the restriction enzymes EcoRI and PstI. Using chromosomal DNA of the budding yeast BY4741 as a template, an SIR2 gene (about 2600 base pairs) comprising promoter and terminator regions was amplified by PCR using the primers MN2515 and MN2503.

Using a plasmid p3009 (NBRP: Yeast ID BYP3009) comprising an HIS3 gene from Candida glabrata (CgHIS3) as a template, the CgHIS3 gene was amplified by PCR using the primers MN2517 and MN2518. Any marker gene apart from CgHIS3 can be used by changing the primer design.

The plasmid pTA-5′-UP_5′+3′ cleaved with EcoRI and PstI was mixed with the above SIR2 gene and CgHIS3 gene (molar ratio about 1:2:2), an equal amount of NEBuilder reagent (New England Biolab) was added, and the mixture was maintained for 1 hour at 50° C. to ligate the three different DNA fragments. The E. Coli DH5? strain was transformed to obtain the target recombinant plasmid (FIG. 15). The primer sequences shown in the following table were used.

TABLE 8

| Primer | Explanation | Sequence |
|---|---|---|
| MN2503 | SIR2_3′ | GAGGCTATACCACCACC |
| | | TCCTTTG |
| MN2509 | 5-UP_5′_Sal | GGAATTATATTTTGAGT |
| | | CGACCCATCTAATGTAC |
| | | TGGTATCAC |
| MN2510 | 5′-UP_5′_Eco | GTACTGCTCTTCCGTTT |
| | | TCAGAATTCAGATGGAC |
| | | CAGCCAAGGAC |
| MN2511 | 5-Up_3_Pst | GAGTAGGGAATATTACT |
| | | GGCTGCAGATAAGTCTT |
| | | GAATGAACGTATACGC |
| MN2512 | 6′-Up_3′_Bam | GGCAAAAAAAGAAAAGC |
| | | TCCGGATCCAGATTGTA |
| | | CGTAAGGTGAGAAGC |
| MN2515 | SIR2 5′ for | TTGGCTGGTCCATCTGA |
| | NEBuilder | ATTCGCGATGATGTTCC |
| | | GATCGGAAGCTC |
| MN2517 | GgHIS3 5′ for | GAGGTGGTGGTATAGCC |
| | NEBuilder | TCGATGCACAGAAACTA |
| | | AGGTACAA |
| MN2518 | GgHIS3 3′ for | TCATTCAAGACTTATCT |
| | NEBuilder | GCAGGCTCTGCTAACTC |
| | | AGTCATATTG |

Bold letters indicate overlapping sequences required for the ligation of each fragment.

The resulting plasmid was cleaved with the restriction enzymes SalI and NotI, purified with a DNA purification kit (Macherey-Nagel, Takara Bio), and used to transform the yeast strain MFY2010SAH (MATα his3Δ1 ura3Δ0 leu2Δ0 met15Δ0 lys2Δ0 fob1Δ0 RDN1-15 BC1-10-URA3), which already has 10 barcodes.

Insertion at the target site was confirmed by extracting chromosomal DNA from the transformed yeast strain, and performing a PCR reaction with the primers MN2509 and MN2512 to obtain a roughly 4,800 bp expected DNA fragment from the recombinant.

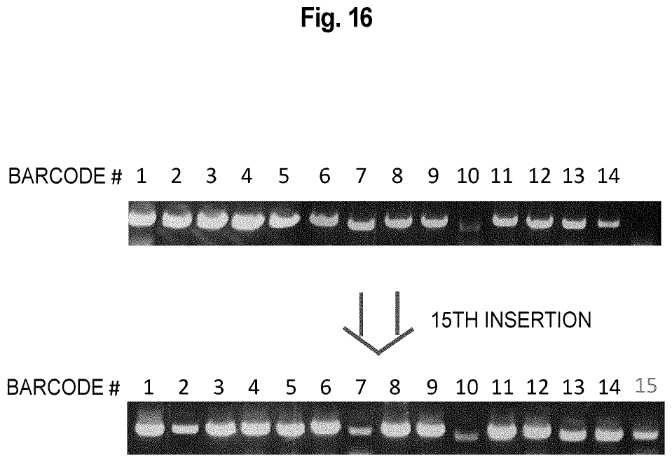

A highly stable artificial chromosome vector (FIG. 16) comprising a total of 15 barcodes (BAC1 to BAC15) was obtained by introducing the SIR2 gene into the budding yeast chromosome 4 by the above methods.

1. An artificial chromosome vector derived from a eukaryotic chromosome, the vector comprising repetitive units of multiple ribosomal RNA genes (rDNA) and intergenic regions between the repetitive units, wherein an intergenic region 1 (IGS1) of the intergenic regions comprises a barcode sequence for incorporating and distinguishing a target gene.

2. The artificial chromosome vector according to claim 1, wherein the intergenic region comprises an autonomously replicating sequence (ARS), a cohesin binding site, a 5S rDNA sequence, a non-coding promoter (E-pro) sequence and a replication fork barrier (RFB) sequence in that order from the 5′ side of the rDNA gene.

3. The artificial chromosome vector according to claim 2, wherein the intergenic region further comprises a condensin binding site.

4. The artificial chromosome vector according to any one of Claims +te 3 claim 1, wherein each barcode sequence is inserted into a restriction enzyme site located between a 5S rDNA sequence and an E-pro sequence.

5. The artificial chromosome vector according to claim 1, further comprising a telomere sequence and a centromere sequence.

6. The artificial chromosome vector according to claim 1, wherein each barcode sequence comprises a promoter sequence and a terminator sequence for controlling expression of the target gene.

7. The artificial chromosome vector according to claim 6, wherein the terminator sequence is disposed on the 3′ side of each target gene and the promoter sequence is disposed on the 5′ side.

8. The artificial chromosome vector according to claim 1, wherein the number of repetitive units is from 2 to 150.

9. The artificial chromosome vector according to claim 1, wherein the eukaryote is a human or yeast.

10. The artificial chromosome vector according to claim 9, wherein the yeast is a budding yeast, and the repetitive units of rDNA are derived from chromosome 12 of the budding yeast.

11. The artificial chromosome vector according to claim 6, wherein the promoter is a galactose inducible (GAL) promoter.

12. The artificial chromosome vector according to claim 1, wherein the inserted target gene is homologous or heterologous to the host into which the artificial chromosome vector is introduced.

13. The artificial chromosome vector according to claim 1, wherein the target genes to be inserted into the barcode sequences are identical or different from each other.

14. A eukaryotic cell comprising the artificial chromosome vector according to claim 1.

15. A method for manufacturing the artificial chromosome vector according to claim 1, the method comprising a step of inserting barcode sequences into IGS1.

16-19. (canceled)

20. A method for manufacturing multiple identical or different target genes or expression products of the target genes, the method comprising:
1) a step of inserting the target genes into the barcode sequences of the artificial chromosome vector according to claim 1, and
2) a step of maintaining and culturing in a host the resulting artificial chromosome vector obtained in the 1).

21-25. (canceled)

26. A method for analyzing behaviors of multiple different target genes derived from heterologous cells, or the expression products of the target genes, in host cells, the method comprising:
1) a step of maintaining and culturing an artificial chromosome vector according to claim 1 which is an artificial chromosome comprising a target gene inserted into a barcode sequence, in host cells, and
2) a step of analyzing a function of the target gene or an expression product thereof, or behaviors thereof in the host cells.

27-32. (canceled)

33. An artificial cell comprising a liposome and the artificial chromosome vector according to claim 1.

34. A method for manufacturing an artificial cell, the method comprising a step of introducing the artificial chromosome vector according to claim 1 into a liposome.
